SEQUENCE LISTING (62 sequences)

SEQ ID NO: 1 - protein, 4 aa, Artificial Sequence
  Synthetic polypeptide
EHVI

SEQ ID NO: 2 - DNA, 30 bp, Artificial Sequence
  Synthetic polynucleotide
ataatcggtcatcatgcttatggggaccag

SEQ ID NO: 3 - protein, 10 aa, Artificial Sequence
  Synthetic polypeptide
IIGHHAYGDQ

SEQ ID NO: 4 - DNA, 63 bp, Artificial Sequence
  Synthetic polynucleotide
agcggatgggtaaaacctattataatcggtcatcatgcttatggggaccagtacagagcaacg

SEQ ID NO: 5 - protein, 21 aa, Artificial Sequence
  Synthetic polypeptide
SGWVKPIIIGHHAYGDQYRAT

SEQ ID NO: 6 - DNA, 57 bp, Artificial Sequence
  Synthetic polynucleotide
tggaagtctccgaacggcaccattcagaatattctcggcggcactgtgttccgggag

SEQ ID NO: 7 - protein, 19 aa, Artificial Sequence
  Synthetic polypeptide
WKSPNGTIQNILGGTVFRE

SEQ ID NO: 8 - DNA, 54 bp, Artificial Sequence
  Synthetic polynucleotide
ggttggacgaaaccaatcactattggcaagcatgctcatggagaccagtacaag

SEQ ID NO: 9 - protein, 18 aa, Artificial Sequence
  Synthetic polypeptide
GWTKPITIGKHAHGDQYK

SEQ ID NO: 10 - DNA, 51 bp, Artificial Sequence
  Synthetic polynucleotide
ttggatattctcgacacagcaggtcgagaggagtacagtgcaatgagggac

SEQ ID NO: 11 - protein, 17 aa, Artificial Sequence
  Synthetic polypeptide
LDILDTAGREEYSAMRD

SEQ ID NO: 12 - DNA, 51 bp, Artificial Sequence
  Synthetic polynucleotide
ttggatattctcgacacagcaggtcatgaggagtacagtgcaatgagggac

SEQ ID NO: 13 - protein, 17 aa, Artificial Sequence
  Synthetic polypeptide
LDILDTAGHEEYSAMRD

SEQ ID NO: 14 - DNA, 51 bp, Artificial Sequence
  Synthetic polynucleotide
ttggatattctcgacacagcaggtaaagaggagtacagtgcaatgagggac

SEQ ID NO: 15 - protein, 17 aa, Artificial Sequence
  Synthetic polypeptide
LDILDTAGKEEYSAMRD

SEQ ID NO: 16 - DNA, 51 bp, Artificial Sequence
  Synthetic polynucleotide
ttggatattctcgacacagcaggtctagaggagtacagtgcaatgagggac

SEQ ID NO: 17 - protein, 17 aa, Artificial Sequence
  Synthetic polypeptide
LDILDTAGLEEYSAMRD

SEQ ID NO: 18 - DNA, 51 bp, Artificial Sequence
  Synthetic polynucleotide
ataggtgattttggtctagctacagagaaatctcgatggagtgggtcccat

SEQ ID NO: 19 - protein, 17 aa, Artificial Sequence
  Synthetic polypeptide
IGDFGLATEKSRWSGSH

SEQ ID NO: 20 - DNA, 51 bp, Artificial Sequence
  Synthetic polynucleotide
cgagatcctctctctgaaatcactgagcaggagaaagattttctatggagt

SEQ ID NO: 21 - protein, 17 aa, Artificial Sequence
  Synthetic polypeptide
RDPLSEITEQEKDFLWS

SEQ ID NO: 22 - DNA, 51 bp, Artificial Sequence
  Synthetic polynucleotide
cgagatcctctctctgaaatcactcagcaggagaaagattttctatggagt

SEQ ID NO: 23 - protein, 16 aa, Artificial Sequence
  Synthetic polypeptide
RDPLSEITQQEKDFLW

SEQ ID NO: 24 - DNA, 51 bp, Artificial Sequence
  Synthetic polynucleotide
cgagatcctctctctgaaatcactaagcaggagaaagattttctatggagt

SEQ ID NO: 25 - protein, 17 aa, Artificial Sequence
  Synthetic polypeptide
RDPLSEITKQEKDFLWS

SEQ ID NO: 26 - DNA, 51 bp, Artificial Sequence
  Synthetic polynucleotide
cgagatcctctctctgaaatcactgcgcaggagaaagattttctatggagt

SEQ ID NO: 27 - protein, 17 aa, Artificial Sequence
  Synthetic polypeptide
RDPLSEITAQEKDFLWS

SEQ ID NO: 28 - DNA, 51 bp, Artificial Sequence
  Synthetic polynucleotide
cgagatcctctctctgaaatcactgggcaggagaaagattttctatggagt

SEQ ID NO: 29 - protein, 17 aa, Artificial Sequence
  Synthetic polypeptide
RDPLSEITGQEKDFLWS

SEQ ID NO: 30 - DNA, 51 bp, Artificial Sequence
  Synthetic polynucleotide
cgagatcctctctctgaaatcactgtgcaggagaaagattttctatggagt

SEQ ID NO: 31 - protein, 17 aa, Artificial Sequence
  Synthetic polypeptide
RDPLSEITVQEKDFLWS

SEQ ID NO: 32 - DNA, 51 bp, Artificial Sequence
  Synthetic polynucleotide
cgagatcctctctctgaaatcactgagccggagaaagattttctatggagt

SEQ ID NO: 33 - protein, 17 aa, Artificial Sequence
  Synthetic polypeptide
RDPLSEITEPEKDFLWS

SEQ ID NO: 34 - DNA, 51 bp, Artificial Sequence
  Synthetic polynucleotide
cgagatcctctctctgaaatcactgagaaggagaaagattttctatggagt

SEQ ID NO: 35 - protein, 17 aa, Artificial Sequence
  Synthetic polypeptide
RDPLSEITEKEKDFLWS

SEQ ID NO: 36 - DNA, 51 bp, Artificial Sequence
  Synthetic polynucleotide
cgagatcctctctctgaaatcactgaggaggagaaagattttctatggagt

SEQ ID NO: 37 - protein, 17 aa, Artificial Sequence
  Synthetic polypeptide
RDPLSEITEEEKDFLWS

SEQ ID NO: 38 - DNA, 51 bp, Artificial Sequence
  Synthetic polynucleotide
cgagatcctctctctgaaatcactgagcgggagaaagattttctatggagt

SEQ ID NO: 39 - protein, 17 aa, Artificial Sequence
  Synthetic polypeptide
RDPLSEITEREKDFLWS

SEQ ID NO: 40 - DNA, 51 bp, Artificial Sequence
  Synthetic polynucleotide
cgagatcctctctctgaaatcactgagctggagaaagattttctatggagt

SEQ ID NO: 41 - protein, 17 aa, Artificial Sequence
  Synthetic polypeptide
RDPLSEITELEKDFLWS

SEQ ID NO: 42 - DNA, 51 bp, Artificial Sequence
  Synthetic polynucleotide
ttcatgaaacaaatgaatgatgcacgtcatggtggctggacaacaaaaatg

SEQ ID NO: 43 - protein, 17 aa, Artificial Sequence
  Synthetic polypeptide
FMKQMNDARHGGWTTKM

SEQ ID NO: 44 - DNA, 51 bp, Artificial Sequence
  Synthetic polynucleotide
ttcatgaaacaaatgaatgatgcacttcatggtggctggacaacaaaaatg

SEQ ID NO: 45 - protein, 17 aa, Artificial Sequence
  Synthetic polypeptide
FMKQMNDALHGGWTTKM

SEQ ID NO: 46 - protein, 7 aa, Artificial Sequence
  Synthetic polypeptide
GGGSGGG

SEQ ID NO: 47 - protein, 8 aa, Artificial Sequence
  Synthetic polypeptide
GGGSGGGG

SEQ ID NO: 48 - protein, 9 aa, Artificial Sequence
  Synthetic polypeptide
GGGGSGGGG

SEQ ID NO: 49 - protein, 8 aa, Artificial Sequence
  Synthetic polypeptide
GGGGSGGG

SEQ ID NO: 50 - protein, 16 aa, Artificial Sequence
  Synthetic polypeptide
IIGGGSGGGGSGGGGS

SEQ ID NO: 51 - protein, 28 aa, Artificial Sequence
  Synthetic polypeptide
MAISGVPVLGFFIIAVLMSAQESWAIKE

SEQ ID NO: 52 - protein, 254 aa, Homo sapiens
MAISGVPVLGFFIIAVLMSAQESWAIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTENVVCALGLTVGLVGIIIGTIFIIKGVRKSNAAERRGPL

SEQ ID NO: 53 - DNA, 690 bp, Artificial Sequence
  Synthetic polynucleotide
atggccataagtggagtccctgtgctaggatttttcatcatagctgtgctgatgagcgctcaggaatcatgggctatcaaagaagaacatgtgatcatacaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacttggaaatcatgacaaagcgctccaactatactccgatcaccaatgacaagttcaccccaccagtggtcaatgtcacgtggcttcgaaatggaaaacctgtcaccacaggagtgtcagagacagtcttcctgcccagggaagaccaccttttccgcaagttccactatctccccttcctgccctcaactgaggacgtttacgactgcagggtggagcactggggcttggatgagcctcttctcaagcactgggagtttgatgctccaagccctctcccagagactacagagaacgtggtgtgtgccctgggcctgactgtgggtctggtgggcatcattattgggaccatcttcatcatcaagggattgcgcaaaagcaatgcagcagaacgcagggggcctctgtaa

SEQ ID NO: 54 - protein, 256 aa, Artificial Sequence
  Synthetic polypeptide
MAISGVPVLGFFIIAVLMSAQESWAIKEEHVIIIGHHAYGDQIIGGGSGGGGSGGGGSIIQAEFYLNPDQSGEFMFDFDGDEIFHVDMAKKETVWRLEEFGRFASFEAQGALANIAVDKANLEIMTKRSNYTPITNDKFTPPVVNVTWLRNGKPVTTGVSETVFLPREDHLFRKFHYLPFLPSTEDVYDCRVEHWGLDEPLLKHWEFDAPSPLPETTENVVCALGLTVGLVGIIIGTIFIIKGLRKSNAAERRGPL

SEQ ID NO: 55 - DNA, 771 bp, Artificial Sequence
  Synthetic polynucleotide
atggccataagtggagtccctgtgctaggatttttcatcatagctgtgctgatgagcgctcaggaatcatgggctatcaaagaagaacatgtgatcataatcggtcatcatgcttatggggaccagatcatcggaggaggaagtggcggcggcggcagcggtggtggtggttcgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacttggaaatcatgacaaagcgctccaactatactccgatcaccaatgacaagttcaccccaccagtggtcaatgtcacgtggcttcgaaatggaaaacctgtcaccacaggagtgtcagagacagtcttcctgcccagggaagaccaccttttccgcaagttccactatctccccttcctgccctcaactgaggacgtttacgactgcagggtggagcactggggcttggatgagcctcttctcaagcactgggagtttgatgctccaagccctctcccagagactacagagaacgtggtgtgtgccctgggcctgactgtgggtctggtgggcatcattattgggaccatcttcatcatcaagggattgcgcaaaagcaatgcagcagaacgcagggggcctctgtaa

SEQ ID NO: 56 - protein, 266 aa, Artificial Sequence
  Synthetic polypeptide
MAISGVPVLGFFIIAVLMSAQESWAIKEEHVISGWVKPIIIGHHAYGDQYRATIIGGGSGGGGSGGGGSIIQAEFYLNPDQSGEFMFDFDGDEIFHVDMAKKETVWRLEEFGRFASFEAQGALANIAVDKANLEIMTKRSNYTPITNDKFTPPVVNVTWLRNGKPVTTGVSETVFLPREDHLFRKFHYLPFLPSTEDVYDCRVEHWGLDEPLLKHWEFDAPSPLPETTENVVCALGLTVGLVGIIIGTIFIIKGLRKSNAAERRGP

SEQ ID NO: 57 - DNA, 804 bp, Artificial Sequence
  Synthetic polynucleotide
atggccataagtggagtccctgtgctaggatttttcatcatagctgtgctgatgagcgctcaggaatcatgggctatcaaagaagaacatgtgatcagcggatgggtaaaacctattataatcggtcatcatgcttatggggaccagtacagagcaacgatcatcggaggaggaagtggcggcggcggcagcggtggtggtggttcgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacttggaaatcatgacaaagcgctccaactatactccgatcaccaatgacaagttcaccccaccagtggtcaatgtcacgtggcttcgaaatggaaaacctgtcaccacaggagtgtcagagacagtcttcctgcccagggaagaccaccttttccgcaagttccactatctccccttcctgccctcaactgaggacgtttacgactgcagggtggagcactggggcttggatgagcctcttctcaagcactgggagtttgatgctccaagccctctcccagagactacagagaacgtggtgtgtgccctgggcctgactgtgggtctggtgggcatcattattgggaccatcttcatcatcaagggattgcgcaaaagcaatgcagcagaacgcagggggcctctgtaa

SEQ ID NO: 58 - protein, 265 aa, Artificial Sequence
  Synthetic polypeptide
MAISGVPVLGFFIIAVLMSAQESWAIKEEHVIWKSPNGTIQNILGGTVFREIIGGGSGGGGSGGGGSIIQAEFYLNPDQSGEFMFDFDGDEIFHVDMAKKETVWRLEEFGRFASFEAQGALANIAVDKANLEIMTKRSNYTPITNDKFTPPVVNVTWLRNGKPVTTGVSETVFLPREDHLFRKFHYLPFLPSTEDVYDCRVEHWGLDEPLLKHWEFDAPSPLPETTENVVCALGLTVGLVGIIIGTIFIIKGLRKSNAAERRGPL

SEQ ID NO: 59 - DNA, 798 bp, Artificial Sequence
  Synthetic polynucleotide
atggccataagtggagtccctgtgctaggatttttcatcatagctgtgctgatgagcgctcaggaatcatgggctatcaaagaagaacatgtgatctggaagtctccgaacggcaccattcagaatattctcggcggcactgtgttccgggagatcatcggaggaggaagtggcggcggcggcagcggtggtggtggttcgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacttggaaatcatgacaaagcgctccaactatactccgatcaccaatgacaagttcaccccaccagtggtcaatgtcacgtggcttcgaaatggaaaacctgtcaccacaggagtgtcagagacagtcttcctgcccagggaagaccaccttttccgcaagttccactatctccccttcctgccctcaactgaggacgtttacgactgcagggtggagcactggggcttggatgagcctcttctcaagcactgggagtttgatgctccaagccctctcccagagactacagagaacgtggtgtgtgccctgggcctgactgtgggtctggtgggcatcattattgggaccatcttcatcatcaagggattgcgcaaaagcaatgcagcagaacgcagggggcctctgtaa

SEQ ID NO: 60 - protein, 264 aa, Artificial Sequence
  Synthetic polypeptide
MAISGVPVLGFFIIAVLMSAQESWAIKEEHVIGWTKPITIGKHAHGDQYKIIGGGSGGGGSGGGGSIIQAEFYLNPDQSGEFMFDFDGDEIFHVDMAKKETVWRLEEFGRFASFEAQGALANIAVDKANLEIMTKRSNYTPITNDKFTPPVVNVTWLRNGKPVTTGVSETVFLPREDHLFRKFHYLPFLPSTEDVYDCRVEHWGLDEPLLKHWEFDAPSPLPETTENVVCALGLTVGLVGIIIGTIFIIKGLRKSNAAERRGPL

SEQ ID NO: 61 - DNA, 795 bp, Artificial Sequence
  Synthetic polynucleotide
atggccataagtggagtccctgtgctaggatttttcatcatagctgtgctgatgagcgctcaggaatcatgggctatcaaagaagaacatgtgatcggttggacgaaaccaatcactattggcaagcatgctcatggagaccagtacaagatcatcggaggaggaagtggcggcggcggcagcggtggtggtggttcgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacttggaaatcatgacaaagcgctccaactatactccgatcaccaatgacaagttcaccccaccagtggtcaatgtcacgtggcttcgaaatggaaaacctgtcaccacaggagtgtcagagacagtcttcctgcccagggaagaccaccttttccgcaagttccactatctccccttcctgccctcaactgaggacgtttacgactgcagggtggagcactggggcttggatgagcctcttctcaagcactgggagtttgatgctccaagccctctcccagagactacagagaacgtggtgtgtgccctgggcctgactgtgggtctggtgggcatcattattgggaccatcttcatcatcaagggattgcgcaaaagcaatgcagcagaacgcagggggcctctgtaa

SEQ ID NO: 62 - protein, 32 aa, Artificial Sequence
  Synthetic polypeptide
MAISGVPVLGFFIIAVLMSAQESWAIKEEHVI

(19) United States
(12) Patent Application Publication
(10) Pub. No.: US 2019/0070275 A1
(43) Pub. Date: Mar. 7, 2019

(54) NEOANTIGEN COMPOSITIONS AND METHODS OF USING THE SAME IN IMMUNOONCOTHERAPY

(71) Applicant: Oceanside Biotechnology, La Jolla, CA (US)

(72) Inventor: Douglas Wayne Ethell, Riverside, CA (US)

(21) Appl. No.: 16/086,556

(22) PCT Filed: Feb. 22, 2017
(86) PCT No.: PCT/US17/18855

§ 371 Date: Sep. 19, 2018

Related U.S. Application Data
(60) Provisional application No. 62/298,275, filed on Feb. 22, 2016.

(51) Int. Cl. A61K 39/00; C07K 14/74; C07K 14/47

(52) U.S. Cl. CPC A61K 39/0011; C07K 14/70539; C07K 14/47; C07K 2319/40; A61K 2039/5154; A61K 2039/605; A61K 2039/5158

(57) ABSTRACT

Provided herein are, inter alia, compositions including a fusion protein containing an antigenic cancer peptide and a mature MHC class II peptide, a nucleic acid encoding the protein, a pharmaceutical formulation thereof, an antigen-presenting cell expressing the protein. Also provided herein are methods for treating cancer utilizing these compositions.

### CROSS-REFERENCES TO RELATED APPLICATIONS

This application claims the priority and the benefit of U.S. Provisional Patent Application No. 62/298,275, filed Feb. 22, 2016, the content of which is incorporated herein by reference in its entirety and for all purposes.

### REFERENCE TO A “SEQUENCE LISTING,” A TABLE, OR A COMPUTER PROGRAM LISTING APPENDIX SUBMITTED AS AN ASCII FILE

The Sequence Listing written in file 49906-503001WO_ST25.TXT, created Feb. 22, 2017, 29,951 bytes, machine format IBM-PC, MS-Windows operating system, is hereby incorporated by reference.

### BACKGROUND

In the last decades, an overwhelming number of studies remarkably contributed to the comprehension of the molecular mechanisms leading to cancer. However, this scientific progress in the molecular oncology field has not been paralleled by a comparable progress in cancer therapy. Surgery and/or radiotherapy are the still the main modality of local treatment of cancer in the majority of patients. However, these treatments are effective only at initial phases of the disease and in particular for certain solid tumors, while they are not effective for distant recurrence of the disease. In some tumor classes, chemotherapeutic treatments have been developed, which generally relies on drugs, hormones and antibodies, targeting specific biological processes used by cancers to grow and spread. However, so far many cancer therapies had limited efficacy due to severity of side effects and overall toxicity. Therefore, there is a great demand for new therapeutics to advance the treatment of cancer. Provided herein are solutions to these and other problems in the art.

### SUMMARY

Provided herein, inter alia, are compositions and methods for treating cancer.

In one aspect, provided herein is a protein that includes an antigenic cancer peptide covalently attached to a mature MHC class II peptide, and the antigenic cancer peptide is capable of non-covalently binding directly to the MHC class II peptide.

In one aspect, provided herein is an antigen-presenting cell. The antigen-presenting cell includes one or more proteins described herein.

In one aspect, provided herein is a nucleic acid encoding any one protein described herein.

In one aspect, provided herein is a pharmaceutical formulation that includes a nucleic acid disclosed herein and a pharmaceutically acceptable excipient.

In yet another aspect, a dendritic cell or B cell that includes any nucleic acid as described herein is provided.

In one aspect, a method of treating cancer in a patient in need thereof is provided. The method includes contacting in vitro the antigen-presenting cell as described herein with a CD4+ T cell, thereby activing the CD4+ T cell, where the CD4+ T cell and the antigen-presenting cell are derived from the patient; allowing the activated CD4+ T cell to expand thereby forming a plurality of expanded CD4+ T cells; and administering to the patient an effective amount of the plurality of expanded CD4+ T cells.

In one aspect, a method of treating cancer in a patient in need thereof is provided. The method includes administering to the patient an effective amount of the nucleic acid as described herein.

### BRIEF DESCRIPTION OF THE DRAWINGS

FIG. 1: a diagram showing autologous T cell transfer for treating tumors.

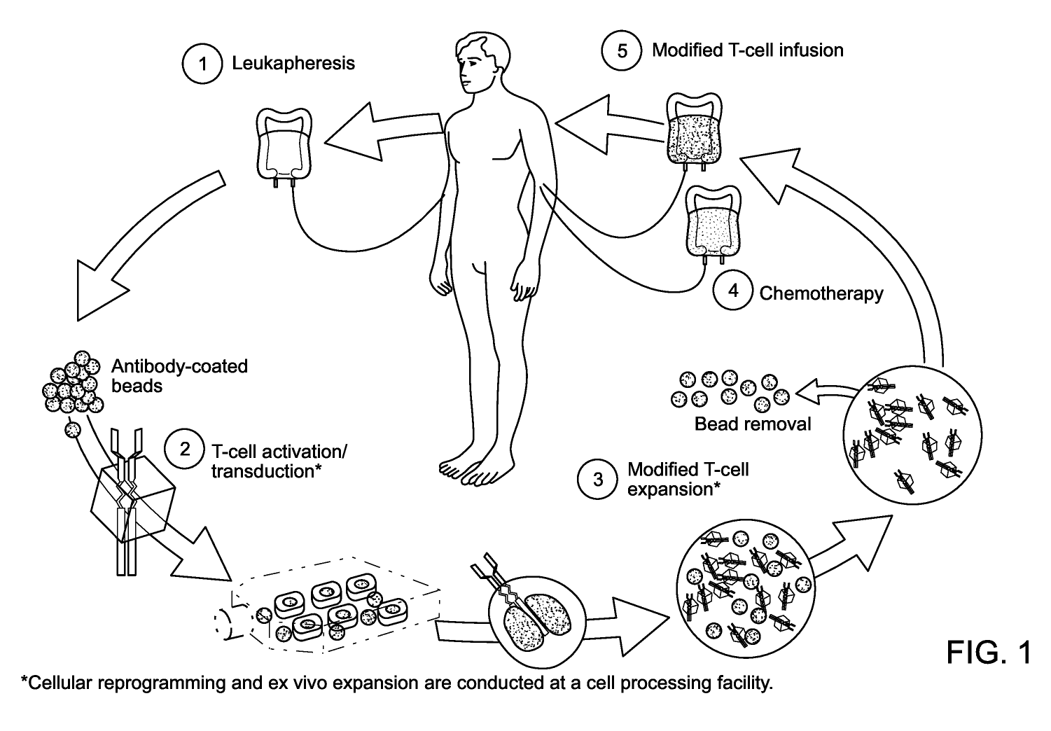

FIGS. 2A-2B. FIG. 2A is a schematic illustration of the fusion protein disclosed herein. FIG. 2B is the predicted 3D structure of the fusion protein.

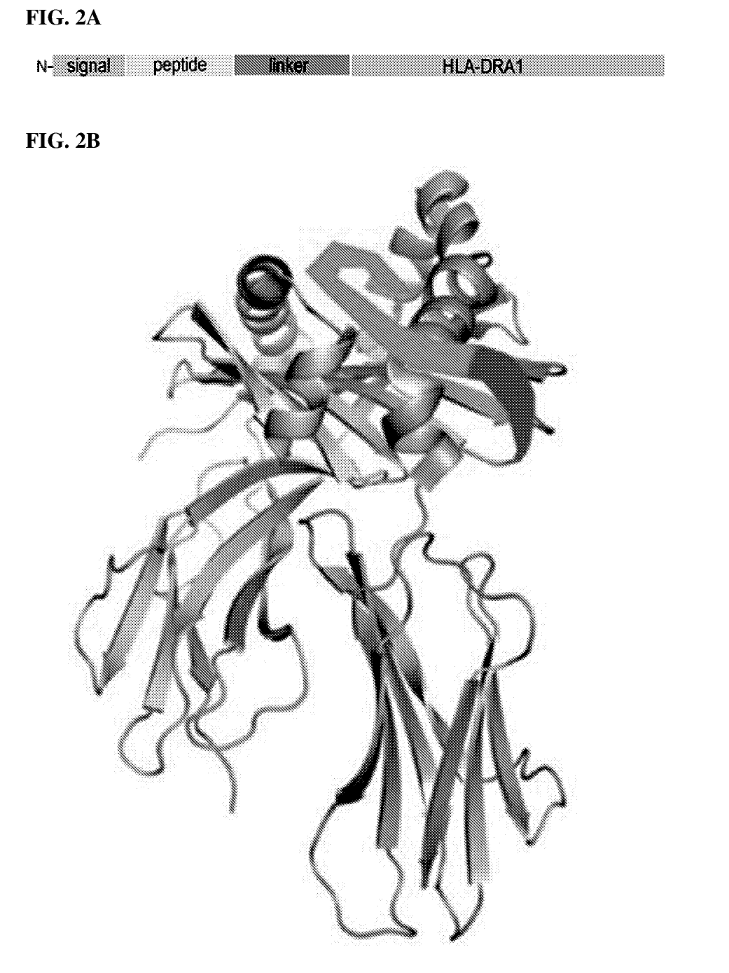

FIG. 3 depicts therapeutic workflow with the platform of the invention.

### DETAILED DESCRIPTION

### I. Definitions

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

The use of a singular indefinite or definite article (e.g., “a,” “an,” “the,” etc.) in this disclosure and in the following claims follows the traditional approach in patents of meaning “at least one” unless in a particular instance it is clear from context that the term is intended in that particular instance to mean specifically one and only one. Likewise, the term “comprising” is open ended, not excluding additional items, features, components, etc. References identified herein are expressly incorporated herein by reference in their entireties unless otherwise indicated.

The terms “comprise,” “include,” and “have,” and the derivatives thereof, are used herein interchangeably as comprehensive, open-ended terms. For example, use of “comprising,” “including,” or “having” means that whatever element is comprised, had, or included, is not the only element encompassed by the subject of the clause that contains the verb.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may In embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the invention may comprise natural nucleic acid sequences and variants thereof, artificial nucleic acid sequences, or a combination of such sequences.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:

1) Alanine (A), Glycine (G);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V);

6) Phenylalanine (F), Tyrosine (Y), Tryptophan (W);

7) Serine (S), Threonine (T); and

8) Cysteine (C), Methionine (M)

(see, e.g., Creighton, Proteins (1984)).

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, optionally 65%, 70%, 75%, 80%, 85%, 90%, 95%, 98%, or 99% identity over a specified region, e.g., of the entire polypeptide sequences of the invention or individual domains of the polypeptides of the invention), when compared and aligned for maximum correspondence over a comparison window, or designated region as measured using one of the following sequence comparison algorithms or by manual alignment and visual inspection. Such sequences are then said to be “substantially identical.” This definition also refers to the complement of a test sequence. Optionally, the identity exists over a region that is at least about 50 nucleotides in length, or more preferably over a region that is 100 to 500 or 1000 or more nucleotides in length. The present invention includes nucleic acids sequences and polypeptides that are substantially identical to any of SEQ ID NOs:1-62.

The term “MHC class II peptide” refers in its customary sense to a protein having a functional antigen-binding groove open at both ends and minimally containing a functional alpha subunit (including functional fragments of a natural alpha subunit) derived from the HLA gene (e.g. an HLA-DRA alpha chain peptide). The antigen-binding groove generally binds to peptide sequences 15 to 24 amino acids in length. A mature MHC class II peptide does not include a full length signal peptide.

A “signal peptide,” as used herein, refers to an amino acid sequence that directs the transport of a protein to the endoplasmic reticulum (ER) for post-translational modification and necessarily includes a protease recognition sequence. The signal peptide is typically at the N-terminal end of a protein and the protease recognition sequence is typically at the C-terminal end of the signal peptide. A “protease recognition sequence” is an amino acid sequence recognized by signal peptidases that typically functions to cleave the signal peptide. In embodiments, the protease recognition sequence is a four amino acid sequence such as EHVI (SEQ ID NO: 1). In embodiments, the mature MHC class II peptide includes a protease recognition sequence but not any other portion of the signal peptide. The protease recognition sequence may be on the N-terminal end of the MHC class II peptide.

In embodiments, the MHC Class II molecule includes an a chain (subunit) and not a β chain (subunit). In other embodiments, the MHC Class II molecule is a heterodimer including an α and β chain. The table below summaries embodiments of MHC class II molecules and their encoding gene(s) for α and β chain.

| MHC class II | | |
|---|---|---|
| molecules | Alpha chain | Beta chain |
| HLA-DM | HLA-DMA | HLA-DMB |
| HLA-DO | HLA-DOA | HLA-DOB |
| HLA-DP | HLA-DPA1 | HLA-DPB1 |
| HLA-DQ | HLA-DQA1, HLA-DQA2 | HLA-DQB1, HLA-DQB2 |
| HLA-DR | HLA-DRA | HLA-DRB1, HLA-DRB3, |
| | | HLA-DRB4, HLA-DRB5 |

The term “antigenic cancer peptide,” “tumor antigen” or “neoantigen,” used herein interchangeably, refers to an antigen (a molecule capable of inducing an immune response in a host subject as a human) derived from a cancer cell or tumor. Antigens derived from tumor cells are referred to herein as tumor-specific antigens (TSAs). In embodiments, a TSA results from a tumor-specific mutation.

For example, where the tumor cell is a breast cancer cell, the antigen may be one of EpCAM (epithelial cell adhesion molecule), Her2/neu (Human Epidermal growth factor Receptor 2), MUC-1, EGFR (epidermal growth factor receptor), TAG-12 (tumor associated glycoprotein 12), IGF1 R (insulin-like growth factor 1 receptor), TACSTD2 (tumor associated calcium signal transducer 2), CD318, CD340, CD104, or N-cadherin.

For example, where the tumor cell is a prostate cancer cell, the antigen may be one of EpCAM, MUC-1, EGFR, PSMA (prostate specific membrane antigen), PSA (prostate specific antigen), TACSTD2, PSCA (prostate stem cell antigen), PCSA (prostate cell surface antigen), CD318, CD104, or N-cadherin.

For example, where the tumor cell is a colorectal cancer cell, the antigen may be one of EpCAM, CD66c, CD66e, CEA (carcinoembryonic antigen), TACSTD2, CK20 (cytokeratin 20), CD104, MUC-1, CD318, or N-cadherin.

For example, where the tumor cell is a lung cancer cell the antigen may be one or CK18, CK19, CEA, EGFR, TACSTD2, CD318, CD1 04, or EpCAM.

For example, where the tumor cell is a pancreatic cancer cell the antigen may be one of HSP70, mHSP70, MUC-1, TACSTD2, CEA, CD104, CD318, N-cadherin, or EpCAM1.

For example, where the tumor cell is an ovarian cancer cell the antigen may be one of MUC-1, TACSTD2, CD318, CD104, N-cadherin, or EpCAM.

For example, where the tumor cell is a bladder cancer cell, the antigen may be one of CD34, CD146, CD62, CD105, CD106, VEGF receptor (vascular endothelial growth factor receptor), MUC-1, TACSTD2, EpCAM, CD318, EGFR, 6B5 or Folate binding receptor.

For example, where the tumor cell is a cancer stem cell, the antigen may be one of CD133, CD135, CD 117, or CD34.

For example, where the tumor cell is a melanoma cancer cell, the antigen may be one of the melanocyte differentiation antigens, oncofetal antigens, tumor specific antigens, SEREX antigens or a combination thereof. Examples of melanocyte differentiation antigens, include but are not limited to tyrosinase, gp75, gp100, MART 1 or TRP-2. Examples of oncofetal antigens include antigens in the MAGE family (MAGE-A1, MAGE-A4), BAGE family, GAGE family or NY-ESO1. Examples of tumor-specific antigens include CDK4 and 13-catenin. Examples of SEREX antigens include D-1 and SSX-2.

Exemplary neoantigens also include, but are not limited to, those listed in the table below.

| Cancer type | Neoantigens |
|---|---|
| Melanoma | BRAF-V600E/D/G/K/M/L |
| | BRAF-L597R/S/V/Q |
| | BRAF-D594V/G/A/E/V/R/Q/S |
| | CTNNB1-S45P/Y/F |
| | KIT-V559D/A |
| | KIT-W557R |
| | KIT-L576P |
| | KIT-K642E |
| | NRAS-Q61P/H/L/R/E/K |
| | NRAS-G13V/A/D/C/R |
| | NRAS-G12V/A/D/C/R/S |
| Non-small cell lung carcinoma | AKT1-E17K |
| (NSCLC) | EGFR-G719A/S/C/D |
| | EGFR-T790M |
| | EGFP-L858R |
| | KRAS-G12S/R/C/A/D |
| | KRAS-Q61R/L/P/K/H |
| | KRAS-G13A/D/C/R/S |
| | NRAS-G12C/S/A/D |
| | NRAS-Q61P/H/R/L/K |
| | PIK3CA-E542K |
| | PIK3CA-E545K/Q/A/G/V |
| | PIK3CA-H1047L/R |
| | PTEN-R159S |
| Acute myeloid leukemia (AML) | IDH1-R132C/G/S/L/P/H |
| | IDH2-R140Q/W/L |
| | IDH2-R172S/K/M |
| | JAK2-V617F |
| | KRAS-Q61R/L/P/K/H |
| | NRAS-Q61P/H/L/R/E/K |
| | NRAS-G13V/A/D/C/R |
| | NRAS-G12V/A/D/C/R/S |
| | KIT-D816V |
| | FLT3-D835E/F/H/N/V/A/Y |
| | FLT3-I836L/M |
| Lung cancer | BRAF-V600E |
| | BRAF-L597V |
| | BRAF-G469A |
| | BRAF-Y472C |
| | BRAF-G466V |
| Breast cancer | AKT1-E17K |
| | PIK3CA-E542K |
| | PIK3CA-E545K/Q/A/G/V |
| | PIK3CA-Q546E/K/P/R/L/N |
| | PIK3CA-H1047L/R |
| | PTEN-R159S |
| Ovarian cancer | BRAF-V600E |
| | BRAF-L597R |
| | KRAS-G12C/R/V/A/D |
| | KRAS-G13A/D/C/R |
| | PI3K3CA-P542K |
| | PIK3CA-E545K/Q/A/G/V |
| | PIK3CA-Q546E/K/P/R/L/N |
| | PIK3CA-H1047L/R |
| | PTEN-P130G/Q |
| Colorectal cancer | AKT1-E17K |
| | BRAF-V600E |
| | BRAF-D594G/V |
| | BRAF-G466V |
| | KRAS-G13S |
| | KRAS-Q61R/L/P/K/H |
| | KRAS-G13A/D/C/R/SV |
| | KRAS-A146P/T/V |
| | NRAS-Q61P/H/L/R/E/K |
| | NRAS-G12V/A/D/C/R/S |
| | PIK3CA-E542K |
| | PIK3CA-E545K/Q/A/G/V |
| | PIK3CA-Q546E/K/P/R/L/N |
| | PIK3CA-H1047L/R |
| | PTEN-R159S |
| | SMAD-R361H/C/S |
| Brain cancer | BRAF-V600E |
| | IDH1-R132G/C/S/L/P/H |
| | IDH2-R172S/M/K |
| | IDH2-R140Q |
| Gastrointestinal stromal tumor | BRAF-V600E |
| | KIT-V559D |
| Thyroid cancer | BRAF-V600E |
| | KRAS-Q61K/L/R/P |
| | KRAS-G12C/R/S/V/D |
| | KRAS-G13A/D/C/R/S |
| | KRAS-A146V |

A skilled artisan could readily recognize that the above neoantigens are represented in the format of “[protein name]-[wild type residue][residue number][residue mutation(s)].” The skilled artisan could also readily determine the sequence of each neoantigen listed above according to the sequence databases available in the art. Exemplary sequences of selective neoantigens include, but are not limited to:

```
IDH1-R132H (short)
ataatcggtcatcatgcttatggggaccag (SEQ ID NO: 2)
I I G H H A Y G D Q (SEQ ID NO: 3)
IDH1-R132H
agcggatgggtaaaacctattataatcggtcatcatgcttatggggaccagtacagagcaacg (SEQ ID NO: 4)
  S G W V K P I I I G H H A Y G D Q Y R A T (SEQ ID NO: 5)
IDH2-R140Q
tggaagtctccgaacggcaccattcagaatattctcggcggcactgtgttccgggag (SEQ ID NO: 6)
  W K S P N G T I Q N I L G G I V F R E (SEQ ID NO: 7)
IDH2-R172K
ggttggacgaaaccaatcactattggcaagcatgctcatggagaccagtacaag (SEQ ID NO: 8)
  G W T K P I T I G K H A H G D Q Y K (SEQ ID NO: 9)
KNRAS-61R
ttggatattctcgacacagcaggtcgagaggagtacagtgcaatgagggac (SEQ ID NO: 10)
  L D I L D T A G R E E Y S A M R D (SEQ ID NO: 11)
KNRAS-61H
ttggatattctcgacacagcaggtcatgaggagtacagtgcaatgagggac (SEQ ID NO: 12)
  L D I L D T A G H E E Y S A M R D (SEQ ID NO: 13)
KNRAS-61K
ttggatattctcgacacagcaggtaaagaggagtacagtgcaatgagggac (SEQ ID NO: 14)
  L D I L D T A G K E E Y S A M R D (SEQ ID NO: 15)
KNRAS-61L
ttggatattctcgacacagcaggtctagaggagtacagtgcaatgagggac (SEQ ID NO: 16)
  L D I L D T A G L E E Y S A M R D (SEQ ID NO: 17)
BRAF-V600E
ataggtgattttggtctagctacagAgaaatctcgatggagtgggtcccat (SEQ ID NO: 18)
  I G D F G L A T E K S R W S G S H (SEQ ID NO: 19)
PIK3CA-E545 WT
cgagatcctctctctgaaatcactgagcaggagaaagattttctatggagt (SEQ ID NO: 20)
  R D P L S E I T E Q E K D F L W S (SEQ ID NO: 21)
PIK3CA-E545Q
cgagatcctctctctgaaatcactCagcaggagaaagattttctatggagt (SEQ ID NO: 22)
  R D P L S E I T Q Q E K D F L W S (SEQ ID NO: 23)
PIK3CA-E545K
cgagatcctctctctgaaatcactaagcaggagaaagattttctatggagt (SEQ ID NO: 24)
  R D P L S E I T K Q E K D F L W S (SEQ ID NO: 25)
PIK3CA-E545A
cgagatcctctctctgaaatcactgcgcaggagaaagattttctatggagt (SEQ ID NO: 26)
  R D P L S E I T A Q E K D F L W S (SEQ ID NO: 27)
PIK3CA-E545G
cgagatcctctctctgaaatcactgggcaggagaaagattttctatggagt (SEQ ID NO: 28)
  R D P L S E I T G Q E K D F L W S (SEQ ID NO: 29)
PIK3CA-E545V
cgagatcctctctctgaaatcactgtgcaggagaaagattttctatggagt (SEQ ID NO: 30)
  R D P L S E I T V Q E K D F L W S (SEQ ID NO: 31)
PIK3CA-Q546P
cgagatcctctctctgaaatcactgagccggagaaagattttctatggagt (SEQ ID NO: 32)
  R D P L S E I T E P E K D F L W S (SEQ ID NO: 33)
PIK3CA-Q546K
cgagatcctctctctgaaatcactgagaaggagaaagattttctatggagt (SEQ ID NO: 34)
  R D P L S E I T E K E K D F L W S (SEQ ID NO: 35)
PIK3CA-Q546E
cgagatcctctctctgaaatcactgaggaggagaaagattttctatggagt (SEQ ID NO: 36)
  R D P L S E I T E E E K D F L W S (SEQ ID NO: 37)
PIK3CA-Q546R
cgagatcctctctctgaaatcactgagcgggagaaagattttctatggagt (SEQ ID NO: 38)
  R D P L S E I T E R E K D F L W S (SEQ ID NO: 39)
PIK3CA-Q546L
cgagatcctctctctgaaatcactgagctggagaaagattttctatggagt (SEQ ID NO: 40)
  R D P L S E I T E L E K D F L W S (SEQ ID NO: 41)
PIK3CA-H1047R
ttcatgaaacaaatgaatgatgcacgtcatggtggctggacaacaaaaatg (SEQ ID NO: 42)
  F M K Q M N D A R H G G W T T K M (SEQ ID NO: 43)
PIK3CA-H1047L
ttcatgaaacaaatgaatgatgcacttcatggtggctggacaacaaaaatg (SEQ ID NO: 44)
  F M K Q M N D A L H G G W T T K M (SEQ ID NO: 45)
```

Most popular approaches used to discover new tumor antigens are based on genome-wide transcription profile or total protein content analyses of tumor. These studies usually lead to the identification of groups of mRNAs and proteins which are differentially expressed in tumors. Validation experiments then follow to eventually single out, among the hundreds of RNAs/proteins identified, the very few that have the potential to become useful markers. Alternatively, deep-sequencing technologies can identify mutations within the protein-coding part of the genome (the exome) and predict potential neoantigens (Science 348: 69-74, 2015). In various illustrative embodiments, methods of identifying a neoantigen include the steps of obtaining a tumor sample from a patient; identifying one or more tumor-specific mutations within expressed genes; identifying corresponding peptides containing each of these mutations; optionally filtering the peptides containing one or more tumor-specific mutations through the use of prediction algorithms or through mass spectrometry analysis; and assessing T-cell recognition of optionally filtered peptides. Additional compositions and methods utilized for identifying and assaying neoantigens are described in the U.S. Pat. No. 9,115,402, contents of which are incorporated herein.

The compositions described herein can be purified. Purified compositions are at least about 60% by weight (dry weight) the compound of interest. Preferably, the preparation is at least about 75%, more preferably at least about 90%, and most preferably at least about 99% or higher by weight the compound of interest. Purity is measured by any appropriate standard method, for example, by High-performance liquid chromatography, polyacrylamide gel electrophoresis.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., spodoptera) and human cells.

The term “antigen presenting cell” (APC) includes “professional antigen presenting cells” that constitutively express MHC class II molecules (e.g., B lymphocytes, monocytes, dendritic cells, Langerhans cells, and activated T cells in humans) as well as other antigen presenting cells that are capable of presenting antigen to T cells. APCs can express the appropriate combination of MHC molecules and costimulatory and/or adhesion molecules known in the art to be sufficient for presentation of antigen to T cells or can be induced or engineered to express such molecules.

As used herein, the term “vector” refers to a nucleic acid molecule capable of transporting another nucleic acid to which it has been linked. One type of vector is a “plasmid”, which refers to a linear or circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions. Additionally, some viral vectors are capable of targeting a particular cells type either specifically or non-specifically. Replication-incompetent viral vectors or replication-defective viral vectors refer to viral vectors that are capable of infecting their target cells and delivering their viral payload, but then fail to continue the typical lytic pathway that leads to cell lysis and death.

A “pharmaceutical composition” is a formulation containing the nucleic acids described herein in a form suitable for administration to a subject. In embodiments, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed nucleic acid) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this invention include powders, sprays, ointments, pastes, creams, lotions, gels, solutions, patches and inhalants. In embodiments, the active nucleic acid is mixed under sterile conditions with a pharmaceutically acceptable carrier, and with any preservatives, buffers, or propellants that are required.

As used herein, the phrase “pharmaceutically acceptable” refers to those compounds, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient. A thorough discussion of pharmaceutically acceptable excipients is available in REMINGTON'S PHARMACEUTICAL SCIENCES (Mack Pub. Co., N.J. 1991). Pharmaceutically acceptable excipients in therapeutic compositions may contain liquids such as water, saline, glycerol and ethanol. Additionally, auxiliary substances, such as wetting or emulsifying agents, pH buffering substances, and the like, may be present in such vehicles.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), and transmucosal administration.

Formulations suitable for oral administration can consist of (a) liquid solutions, such as an effective amount of the packaged nucleic acid suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

Pharmaceutical compositions can also include large, slowly metabolized macromolecules such as proteins, polysaccharides such as chitosan, polylactic acids, polyglycolic acids and copolymers (such as latex functionalized sepharose™, agarose, cellulose, and the like), polymeric amino acids, amino acid copolymers, and lipid aggregates (such as oil droplets or liposomes). Additionally, these carriers can function as immunostimulating agents (i.e., adjuvants).

Suitable formulations for rectal administration include, for example, suppositories, which consist of the packaged nucleic acid with a suppository base. Suitable suppository bases include natural or synthetic triglycerides or paraffin hydrocarbons. In addition, it is also possible to use gelatin rectal capsules which consist of a combination of the compound of choice with a base, including, for example, liquid triglycerides, polyethylene glycols, and paraffin hydrocarbons.

Formulations suitable for parenteral administration, such as, for example, by intraarticular (in the joints), intravenous, intramuscular, intratumoral, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Parenteral administration, oral administration, and intravenous administration are the preferred methods of administration. The formulations of compounds can be presented in unit-dose or multi-dose sealed containers, such as ampules and vials.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

A pharmaceutical composition of the invention can be administered to a subject in many of the well-known methods currently used for chemotherapeutic treatment. For example, for treatment of cancers, a composition of the invention may be injected directly into tumors, injected into the blood stream or body cavities or taken orally or applied through the skin with patches. The dose chosen should be sufficient to constitute effective treatment but not so high as to cause unacceptable side effects. The state of the disease condition (e.g., cancer, precancer, and the like) and the health of the patient should preferably be closely monitored during and for a reasonable period after treatment.

As used herein, “monotherapy” refers to the administration of a single active or therapeutic compound to a subject in need thereof. Preferably, monotherapy will involve administration of a therapeutically effective amount of an active composition (e.g., nucleic acid). For example, described herein can be a cancer monotherapy with one of the nucleic acids of the present invention to a subject in need of treatment of cancer. Monotherapy may be contrasted with combination therapy, in which a combination of multiple active compositions (e.g., multiple nucleic acids) is administered, preferably with each component of the combination present in a therapeutically effective amount. Monotherapy with a composition of the present invention may be more effective than combination therapy in inducing a desired biological effect.

As used herein, “combination therapy” or “co-therapy” includes the administration of a composition of the present invention and at least a second agent as part of a specific treatment regimen intended to provide the beneficial effect from the co-action of these therapeutic agents. The beneficial effect of the combination may include, but is not limited to, pharmacokinetic or pharmacodynamic co-action resulting from the combination of therapeutic agents. Administration of these therapeutic agents in combination typically is carried out over a defined time period (usually minutes, hours, days or weeks depending upon the combination selected). “Combination therapy” may be, but generally is not, intended to encompass the administration of two or more of these therapeutic agents as part of separate monotherapy regimens that incidentally and arbitrarily result in the combinations of the present invention.

“Combination therapy” is intended to embrace administration of these therapeutic agents in a sequential manner, wherein each therapeutic agent is administered at a different time, as well as administration of these therapeutic agents, or at least two of the therapeutic agents, in a substantially simultaneous manner Substantially simultaneous administration can be accomplished, for example, by administering to the subject a single capsule having a fixed ratio of each therapeutic agent or in multiple, single capsules for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. The sequence in which the therapeutic agents are administered is not narrowly critical.

“Combination therapy” also embraces the administration of the therapeutic agents as described above in further combination with other biologically active ingredients and non-drug therapies (e.g., surgery or radiation treatment). Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

A composition of the present invention may be administered in combination with a second chemotherapeutic agent. The second chemotherapeutic agent (also referred to as an anti-neoplastic agent or anti-proliferative agent) can be an alkylating agent; an antibiotic; an anti-metabolite; a detoxifying agent; an interferon; a polyclonal or monoclonal antibody; an EGFR inhibitor; a HER2 inhibitor; a histone deacetylase inhibitor; a hormone; a mitotic inhibitor; an MTOR inhibitor; a multi-kinase inhibitor; a serine/threonine kinase inhibitor; a tyrosine kinase inhibitors; a VEGF/VEGFR inhibitor; a taxane or taxane derivative, an aromatase inhibitor, an anthracycline, a microtubule targeting drug, a topoisomerase poison drug, an inhibitor of a molecular target or enzyme (e.g., a kinase or a protein methyltransferase), a cytidine analogue drug or any chemotherapeutic, anti-neoplastic or anti-proliferative agent listed in www.cancer.org/docroot/cdg/cdg_0.asp.

As used herein, a “subject in need thereof” or “a patient” is a subject having cancer. A subject in need thereof can have a precancerous condition. Typically, a subject in need thereof has cancer. A “subject” or a “patient” includes a mammal. The mammal can be e.g., a human or appropriate non-human mammal, such as primate, mouse, rat, dog, cat, cow, horse, goat, camel, sheep or a pig. The subject can also be a bird or fowl. In embodiments, the mammal is a human. Thus the methods are applicable to both human therapy and veterinary applications.

As used herein, the term “cancer” refers to all types of cancer, neoplasm or malignant tumors found in mammals, including leukemias, lymphomas, melanomas, neuroendocrine tumors, carcinomas and sarcomas. Exemplary cancers that may be treated with a composition, pharmaceutical composition, or method provided herein include lymphoma, sarcoma, bladder cancer, bone cancer, brain tumor, cervical cancer, colon cancer, esophageal cancer, gastric cancer, head and neck cancer, kidney cancer, myeloma, thyroid cancer, leukemia, prostate cancer, breast cancer (e.g. triple negative, ER positive, ER negative, chemotherapy resistant, herceptin resistant, HER2 positive, doxorubicin resistant, tamoxifen resistant, ductal carcinoma, lobular carcinoma, primary, metastatic), ovarian cancer, pancreatic cancer, liver cancer (e.g., hepatocellular carcinoma) , lung cancer (e.g. non-small cell lung carcinoma, squamous cell lung carcinoma, adenocarcinoma, large cell lung carcinoma, small cell lung carcinoma, carcinoid, sarcoma), glioblastoma multiforme, glioma, melanoma, prostate cancer, castration-resistant prostate cancer, breast cancer, triple negative breast cancer, glioblastoma, ovarian cancer, lung cancer, squamous cell carcinoma (e.g., head, neck, or esophagus), colorectal cancer, leukemia, acute myeloid leukemia, lymphoma, B cell lymphoma, or multiple myeloma. Additional examples include, cancer of the thyroid, endocrine system, brain, breast, cervix, colon, head & neck, esophagus, liver, kidney, lung, non-small cell lung, melanoma, mesothelioma, ovary, sarcoma, stomach, uterus or Medulloblastoma, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, Paget's Disease of the Nipple, Phyllodes Tumors, Lobular Carcinoma, Ductal Carcinoma, cancer of the pancreatic stellate cells, cancer of the hepatic stellate cells, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a composition, pharmaceutical composition, or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

The term “sarcoma” generally refers to a tumor which is made up of a substance like the embryonic connective tissue and is generally composed of closely packed cells embedded in a fibrillar or homogeneous substance. Sarcomas that may be treated with a composition, pharmaceutical composition, or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a composition, pharmaceutical composition, or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a composition, pharmaceutical composition, or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, ductal carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lobular carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tubular carcinoma, tuberous carcinoma, verrucous carcinoma, or carcinoma villosum.

As used herein, the terms “metastasis,” “metastatic,” and “metastatic cancer” can be used interchangeably and refer to the spread of a proliferative disease or disorder, e.g., cancer, from one organ or another non-adjacent organ or body part. Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

A cancer that is to be treated can be staged according to the American Joint Committee on Cancer (AJCC) TNM classification system, where the tumor (T) has been assigned a stage of TX, T1, T1mic, T1a, T1b, T1c, T2, T3, T4, T4a, T4b, T4c, or T4d; and where the regional lymph nodes (N) have been assigned a stage of NX, N0, N1, N2, N2a, N2b, N3, N3a, N3b, or N3c; and where distant metastasis (M) can be assigned a stage of MX, M0, or M1. A cancer that is to be treated can be staged according to an American Joint Committee on Cancer (AJCC) classification as Stage I, Stage IIA, Stage IIB, Stage IIIA, Stage IIIB, Stage IIIC, or Stage IV. A cancer that is to be treated can be assigned a grade according to an AJCC classification as Grade GX (e.g., grade cannot be assessed), Grade 1, Grade 2, Grade 3 or Grade 4. A cancer that is to be treated can be staged according to an AJCC pathologic classification (pN) of pNX, pN0, PN0 (I−), PN0 (I+), PN0 (mol−), PN0 (mol+), PN1, PN1(mi), PN1a, PN1b, PN1c, pN2, pN2a, pN2b, pN3, pN3a, pN3b, or pN3c.

A cancer that is to be treated can include a tumor that has been determined to be less than or equal to about 2 centimeters in diameter. A cancer that is to be treated can include a tumor that has been determined to be from about 2 to about 5 centimeters in diameter. A cancer that is to be treated can include a tumor that has been determined to be greater than or equal to about 3 centimeters in diameter. A cancer that is to be treated can include a tumor that has been determined to be greater than 5 centimeters in diameter. A cancer that is to be treated can be classified by microscopic appearance as well differentiated, moderately differentiated, poorly differentiated, or undifferentiated. A cancer that is to be treated can be classified by microscopic appearance with respect to mitosis count (e.g., amount of cell division) or nuclear pleiomorphism (e.g., change in cells). A cancer that is to be treated can be classified by microscopic appearance as being associated with areas of necrosis (e.g., areas of dying or degenerating cells). A cancer that is to be treated can be classified as having an abnormal karyotype, having an abnormal number of chromosomes, or having one or more chromosomes that are abnormal in appearance. A cancer that is to be treated can be classified as being aneuploid, triploid, tetraploid, or as having an altered ploidy. A cancer that is to be treated can be classified as having a chromosomal translocation, or a deletion or duplication of an entire chromosome, or a region of deletion, duplication or amplification of a portion of a chromosome.

A cancer that is to be treated can be evaluated by DNA cytometry, flow cytometry, or image cytometry. A cancer that is to be treated can be typed as having about 10%, 20%, 30%, 40%, 50%, 60%, 70%, 80%, or 90% of cells in the synthesis stage of cell division (e.g., in S phase of cell division). A cancer that is to be treated can be typed as having a low S-phase fraction or a high S-phase fraction.

“An effective amount” or “a therapeutically effective amount” as provided herein refers to an amount effective to achieve its intended purpose. The actual amount effective for a particular application will depend, inter alia, on the condition being treated. When administered in methods to treat a disease, the pharmaceutical compositions described herein will contain an amount of active nucleic acid or expanded CD4+ T cells effective to achieve the desired result, e.g., eliciting immune response against tumor, and/or reducing, eliminating, or slowing the progression of disease symptoms (e.g., cancer), or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician. In a preferred aspect, the disease or condition to be treated is cancer.

As used herein, “treating” or “treat” describes the management and care of a patient for the purpose of combating a disease, condition, or disorder and includes the administration of a composition of the present invention to alleviate the symptoms or complications of a disease, condition or disorder, or to eliminate the disease, condition or disorder. The term “treat” can also include treatment of a cell in vitro or an animal model.

As used herein, the term “alleviate” is meant to describe a process by which the severity of a sign or symptom of a disorder is decreased. Importantly, a sign or symptom can be alleviated without being eliminated. The administration of compositions or pharmaceutical compositions of the invention may or can lead to the elimination of a sign or symptom, however, elimination is not required. Effective dosages should be expected to decrease the severity of a sign or symptom. For instance, a sign or symptom of a disorder such as cancer, which can occur in multiple locations, is alleviated if the severity of the cancer is decreased within at least one of multiple locations.

As used herein, the term “severity” is meant to describe the potential of cancer to transform from a precancerous, or benign, state into a malignant state. Alternatively, or in addition, severity is meant to describe a cancer stage, for example, according to the TNM system (accepted by the International Union Against Cancer (UICC) and the American Joint Committee on Cancer (AJCC)) or by other art-recognized methods. Cancer stage refers to the extent or severity of the cancer, based on factors such as the location of the primary tumor, tumor size, number of tumors, and lymph node involvement (spread of cancer into lymph nodes). Alternatively, or in addition, severity is meant to describe the tumor grade by art-recognized methods (see, National Cancer Institute, www.cancer.gov). Tumor grade is a system used to classify cancer cells in terms of how abnormal they look under a microscope and how quickly the tumor is likely to grow and spread. Many factors are considered when determining tumor grade, including the structure and growth pattern of the cells. The specific factors used to determine tumor grade vary with each type of cancer. Severity also describes a histologic grade, also called differentiation, which refers to how much the tumor cells resemble normal cells of the same tissue type (see, National Cancer Institute, website at www.cancer.gov). Furthermore, severity describes a nuclear grade, which refers to the size and shape of the nucleus in tumor cells and the percentage of tumor cells that are dividing (see, National Cancer Institute, www.cancer.gov).

Severity can also describe the degree to which a tumor has secreted growth factors, degraded the extracellular matrix, become vascularized, lost adhesion to juxtaposed tissues, or metastasized. Moreover, severity can describe the number of locations to which a primary tumor has metastasized. Finally, severity can include the difficulty of treating tumors of varying types and locations. For example, inoperable tumors, those cancers which have greater access to multiple body systems (hematological and immunological tumors), and those which are the most resistant to traditional treatments are considered most severe. In these situations, prolonging the life expectancy of the subject and/or reducing pain, decreasing the proportion of cancerous cells or restricting cells to one system, and improving cancer stage/tumor grade/histological grade/nuclear grade are considered alleviating a sign or symptom of the cancer.

As used herein the term “symptom” is defined as an indication of disease, illness, injury, or that something is not right in the body. Symptoms are felt or noticed by the individual experiencing the symptom, but may not easily be noticed by others. Others are defined as non-health-care professionals.

As used herein the term “sign” is also defined as an indication that something is not right in the body. But signs are defined as things that can be seen by a doctor, nurse, or other health care professional.

Cancer is a group of diseases that may cause almost any sign or symptom. The signs and symptoms will depend on where the cancer is, the size of the cancer, and how much it affects the nearby organs or structures. If a cancer spreads (metastasizes), then symptoms may appear in different parts of the body. For example, a cancer may also cause symptoms such as fever, fatigue, or weight loss. Pain may be an early symptom with some cancers such as bone cancers or testicular cancer. But most often pain is a symptom of advanced disease. Along with cancers of the skin, some internal cancers can cause skin signs that can be seen. These changes include the skin looking darker (hyperpigmentation), yellow (jaundice), or red (erythema); itching; or excessive hair growth.

Alternatively, or in addition, cancer subtypes present specific signs or symptoms. Changes in bowel habits or bladder function could indicate cancer. Long-term constipation, diarrhea, or a change in the size of the stool may be a sign of colon cancer. Pain with urination, blood in the urine, or a change in bladder function (such as more frequent or less frequent urination) could be related to bladder or prostate cancer.

Changes in skin condition or appearance of a new skin condition could indicate cancer. Skin cancers may bleed and look like sores that do not heal. A long-lasting sore in the mouth could be an oral cancer, especially in patients who smoke, chew tobacco, or frequently drink alcohol. Sores on the penis or vagina may either be signs of infection or an early cancer.

Unusual bleeding or discharge could indicate cancer. Unusual bleeding can happen in either early or advanced cancer. Blood in the sputum (phlegm) may be a sign of lung cancer. Blood in the stool (or a dark or black stool) could be a sign of colon or rectal cancer. Cancer of the cervix or the endometrium (lining of the uterus) can cause vaginal bleeding. Blood in the urine may be a sign of bladder or kidney cancer. A bloody discharge from the nipple may be a sign of breast cancer.

A thickening or lump in the breast or in other parts of the body could indicate the presence of a cancer. Many cancers can be felt through the skin, mostly in the breast, testicle, lymph nodes (glands), and the soft tissues of the body. A lump or thickening may be an early or late sign of cancer. Any lump or thickening could be indicative of cancer, especially if the formation is new or has grown in size.

Indigestion or trouble swallowing could indicate cancer. While these symptoms commonly have other causes, indigestion or swallowing problems may be a sign of cancer of the esophagus, stomach, or pharynx (throat).

Recent changes in a wart or mole could be indicative of cancer. Any wart, mole, or freckle that changes in color, size, or shape, or loses its definite borders indicates the potential development of cancer. For example, the skin lesion may be a melanoma.

A persistent cough or hoarseness could be indicative of cancer. A cough that does not go away may be a sign of lung cancer. Hoarseness can be a sign of cancer of the larynx (voice box) or thyroid.

While the signs and symptoms listed above are the more common ones seen with cancer, there are many others that are less common and are not listed here.

Treating cancer may result in or can result in a reduction in size of a tumor. A reduction in size of a tumor may also be referred to as “tumor regression”. Preferably, after treatment, tumor size would be reduced by about 5% or greater relative to its size prior to treatment; more preferably, tumor size is reduced by about 10% or greater; more preferably, reduced by about 20% or greater; more preferably, reduced by about 30% or greater; more preferably, reduced by about 40% or greater; even more preferably, reduced by about 50% or greater; and most preferably, reduced by greater than about 75% or greater. Size of a tumor may be measured by any reproducible means of measurement. The size of a tumor may be measured as a diameter of the tumor.

Treating cancer may result in or can result in a reduction in tumor volume. Preferably, after treatment, tumor volume would be reduced by about 5% or greater relative to its size prior to treatment; more preferably, tumor volume is reduced by about 10% or greater; more preferably, reduced by about 20% or greater; more preferably, reduced by about 30% or greater; more preferably, reduced by about 40% or greater; even more preferably, reduced by about 50% or greater; and most preferably, reduced by greater than about 75% or greater. Tumor volume may be measured by any reproducible means of measurement.

Treating cancer may result in or can result in a decrease in number of tumors. Preferably, after treatment, tumor number would be reduced by about 5% or greater relative to number prior to treatment; more preferably, tumor number is reduced by about 10% or greater; more preferably, reduced by about 20% or greater; more preferably, reduced by about 30% or greater; more preferably, reduced by about 40% or greater; even more preferably, reduced by about 50% or greater; and most preferably, reduced by greater than about 75%. Number of tumors may be measured by any reproducible means of measurement. The number of tumors may be measured by counting tumors visible to the naked eye or at a specified magnification. Preferably, the specified magnification is 2×, 3×, 4×, 5×, 10×, or 50×.

Treating cancer may result in or can result in a decrease in number of metastatic lesions in other tissues or organs distant from the primary tumor site. Preferably, after treatment, the number of metastatic lesions would be reduced by about 5% or greater relative to number prior to treatment; more preferably, the number of metastatic lesions is reduced by about 10% or greater; more preferably, reduced by about 20% or greater; more preferably, reduced by about 30% or greater; more preferably, reduced by about 40% or greater; even more preferably, reduced by about 50% or greater; and most preferably, reduced by greater than about 75%. The number of metastatic lesions may be measured by any reproducible means of measurement. The number of metastatic lesions may be measured by counting metastatic lesions visible to the naked eye or at a specified magnification. Preferably, the specified magnification is 2×, 3×, 4×, 5×, 10×, or 50×.

Treating cancer may result in or can result in an increase in average survival time of a population of treated subjects in comparison to a population receiving carrier alone. Preferably, the average survival time would be increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active composition. An increase in average survival time of a population may also be measured, for example, by calculating for a population the average length of survival following completion of a first round of treatment with an active composition.

Treating cancer may result in or can result in an increase in average survival time of a population of treated subjects in comparison to a population of untreated subjects. Preferably, the average survival time would be increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active composition. An increase in average survival time of a population may also be measured, for example, by calculating for a population the average length of survival following completion of a first round of treatment with an active composition.

Treating cancer may result in or can result in increase in average survival time of a population of treated subjects in comparison to a population receiving monotherapy with a drug that is not a composition of the present invention. Preferably, the average survival time would be increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active composition. An increase in average survival time of a population may also be measured, for example, by calculating for a population the average length of survival following completion of a first round of treatment with an active composition.

Treating cancer may result in or can result in a decrease in the mortality rate of a population of treated subjects in comparison to a population receiving carrier alone. Treating cancer may result in or can result in a decrease in the mortality rate of a population of treated subjects in comparison to an untreated population. Treating cancer may result in or can result in a decrease in the mortality rate of a population of treated subjects in comparison to a population receiving monotherapy with a drug that is not a composition of the present invention, or a pharmaceutically acceptable salt, prodrug, metabolite, analog or derivative thereof. Preferably, the mortality rate would be decreased by more than 2%; more preferably, by more than 5%; more preferably, by more than 10%; and most preferably, by more than 25%. A decrease in the mortality rate of a population of treated subjects may be measured by any reproducible means. A decrease in the mortality rate of a population may be measured, for example, by calculating for a population the average number of disease-related deaths per unit time following initiation of treatment with an active composition. A decrease in the mortality rate of a population may also be measured, for example, by calculating for a population the average number of disease-related deaths per unit time following completion of a first round of treatment with an active composition.

Treating cancer may result in or can result in a decrease in tumor growth rate. Preferably, after treatment, tumor growth rate would be reduced by at least 5% relative to number prior to treatment; more preferably, tumor growth rate would be reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Tumor growth rate may be measured by any reproducible means of measurement. Tumor growth rate can be measured according to a change in tumor diameter per unit time.

Treating cancer may result in or can result in a decrease in tumor regrowth. Preferably, after treatment, tumor regrowth would be less than 5%; more preferably, tumor regrowth would be less than 10%; more preferably, less than 20%; more preferably, less than 30%; more preferably, less than 40%; more preferably, less than 50%; even more preferably, less than 50%; and most preferably, less than 75%. Tumor regrowth may be measured by any reproducible means of measurement. Tumor regrowth is measured, for example, by measuring an increase in the diameter of a tumor after a prior tumor shrinkage that followed treatment. A decrease in tumor regrowth is indicated by failure of tumors to reoccur after treatment has stopped.

### II. Compositions

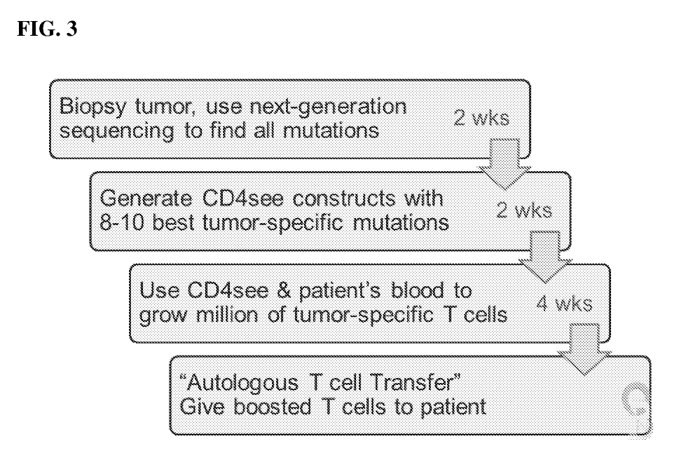

Provided herein is a novel immunotherapy that boosts the ability of endogenous T cells to destroy cancer cells. A rationale for immunotherapy is that cancer cells are replete with potential antigens, which become immunogenic when presented by antigen-presenting cells to helper and cytotoxic T cells. Compared to the existing immunotherapies for cancer, the immunotherapy platform (e.g., compositions and methods) described herein provides a number of benefits and advantages. For example, the platform (e.g., compositions and methods) described herein results in stimulation of antigen-specific CD4+ T cells populations with a much greater efficiency (e.g., 10 times greater or more than other immunotherapies). The platform provided herein also makes it possible to have a rapid development for both known and unknown neoantigens. The platform described herein is amenable to multiplexing, for example, patients can receive more than 1 protein/nucleic acid (e.g., 2, 3, 4, 5, 6, 7, 8, 9, 10, 11, 12, 13, 14, 15, 16, 17, 18, 19, 20 or more proteins/nucleic acids described herein) for the treatment. The platform described herein is also easily scalable, for example, the protein/nucleic acid/vector/pharmaceutical composition described herein can be made in bulk and ready to use. The platform described herein is easy and convenient to carry out, thus providing an excellent amenable personalized treatment for each individual patient. An exemplary therapeutic workflow for such personalized treatment with the platform of the invention is illustrated in FIG. 3. The platform described herein also avoids many potential drawbacks and challenges that other immunotherapies are facing. For example, the platform does not trigger genomic integration because the nucleic acid of the invention is typically packaged with liposome for administration. The platform also has no off-target effects because only the specific neoantigen peptide sequence inserted in the construct/vector is presented. The platform also shows no increase of risk for autoimmunity. The platform utilizes optimized translation, thus reducing the chance of alternative start reads.

Accordingly, in one aspect, provided herein is a protein (or a fusion protein) that includes an antigenic cancer peptide (i.e., a neoantigen) covalently attached to a mature MHC class II peptide, and the antigenic cancer peptide is capable of non-covalently binding directly to the MHC class II peptide. The construct that includes one or more of the fusion proteins described herein is referred to a CD4see construct.

In embodiments, the antigenic cancer peptide is N-terminal to the mature MHC class II peptide.

In embodiments, the protein also includes a peptide linker covalently linking the antigenic cancer peptide and the mature MHC class II peptide. Typically, the peptide linker has one or more (e.g., 1, 2, 3, 4, 5 or more) glycine amino acids, one or more (e.g., 1, 2, 3, 4, 5 or more) serine amino acids, or a combination of one or more (e.g., 1, 2, 3, 4, 5 or more) glycine amino acids and one or more (e.g., 1, 2, 3, 4, 5 or more) serine amino acids. For example, a linker peptide includes a sequence of GGGSGGG (SEQ ID NO: 46), GGGSGGGG (SEQ ID NO: 47), GGGGSGGGG (SEQ ID NO: 48), GGGGSGGG (SEQ ID NO: 49) or IIGGGSGGGGSGGGGS (SEQ ID NO: 50).

In embodiments, the nucleic acid sequence of a linker according to the invention has a degree of sequence identity with one of SEQ ID Nos 46 to 50, that is at least 80%, 81%, 82%, 83%, 84%, 85%, 86%, 87%, 88%, 89%, 90%, 91%, 92%, 93%, 94%, 95%, 96%, 97%, 98%, 99%, or 100%.

In embodiments, the protein also includes a signal peptide covalently attached to the N-terminus of the antigenic cancer peptide. Typically, the signal peptide has 10-30 amino acids in length. In embodiments, the signal peptide includes a sequence of MAISGVPVLGFFIIAVLMSAQESWAIKE (SEQ ID NO: 51). In embodiments, the signal peptide includes a sequence of MAISGVPVLGFFIIAVLMSAQESWAIKEEHVI (SEQ ID NO: 62).

In embodiments, the amino acid sequence of a signal peptide according to the invention has a degree of sequence identity with SEQ ID No 51 or 62, that is at least 80%, 81%, 82%, 83%, 84%, 85%, 86%, 87%, 88%, 89%, 90%, 91%, 92%, 93%, 94%, 95%, 96%, 97%, 98%, 99%, or 100%.

In embodiments, the signal peptide includes a protease recognition sequence at the C-terminus of the signal peptide. For example, the amino acid sequence of this protease recognition sequence comprises SEQ ID NO: 1. In embodiments, the amino acid sequence of this protease recognition sequence according to the invention has a degree of sequence identity with SEQ ID No: 1, that is at least 95%, 96%, 97%, 98%, 99%, or 100%.

In embodiments, the mature MHC class II peptide is a mature HLA-DRA alpha chain peptide. In embodiments, the mature HLA-DRA alpha chain peptide comprises a transmembrane domain and/or a cytoplasmic domain.

The HLA-DRA alpha chain is approximately 33-35 kDa and its gene contains five exons. Exon 1 encodes the leader peptide, exons 2 and 3 encode the two extracellular domains, and exon 4 encodes the transmembrane domain and the cytoplasmic tail. HLA-DRA*01:01 is the predominant allele in the human population and two minor variants are HLA-DRA*01:02:01 and HLA-DRA*01:02:01. These three alleles do not have polymorphisms in the peptide binding part and acts as the sole alpha chain for the beta chains DRB1, DRB3, DRB4 and DRB5. HLA-DRA sequences are publicly available. For example, nucleotide sequences can be found at: NC_000006.12, NC_018917.2, NT_007592.16, NT_113891.3, NT_167245.2, NT_167246.2, NT_167247.2, NT_167248.2, NT_167249.2, NT_187692.1. For example, amino acid sequences can be found at: NP 061984.2.

In embodiments, the mature HLA-DRA alpha chain includes the following amino acid sequence (NCBI Accession No. CAG33294.1):

| (SEQ ID NO: 52) | |
|---|---|
| 1 | maisgvpvlg ffiiavlmsa qeswaikeeh viiqaefyln pdqsgefmfd fdgdeifhvd |
| 61 | makketvwrl eefgrfasfe aqgalaniav dkanleimtk rsnytpitnv ppevtvltns |
| 121 | pvelrepnvl icfidkftpp vvnvtwlrng kpvttgvset vflpredhlf rkfhylpflp |
| 181 | stedvydcvy ehwgldepll khwefdapsp lpettenvvc algltvglvg iiigtifiik |
| 241 | gvrksnaaer rgpl |

Underlined: Signal peptide

In embodiments, the mature HLA-DRA alpha chain includes the following nucleotide sequence:

(SEQ ID NO: 53)
atggccataagtggagtccctgtgctaggatttttcatcatagctgtgct
gatgagcgctcaggaatcatgggctatcaaagaagaacatgtgatcatac
aggccgagttctatctgaatcctgaccaatcaggcgagtttatgtttgac
tttgatggtgatgagattttccatgtggatatggcaaagaaggagacggt
ctggcggcttgaagaatttggacgatttgccagctttgaggctcaaggtg
cattggccaacatagctgtggacaaagccaacttggaaatcatgacaaag
cgctccaactatactccgatcaccaatgacaagttcaccccaccagtggt
caatgtcacgtggcttcgaaatggaaaacctgtcaccacaggagtgtcag
agacagtcttcctgcccagggaagaccaccttttccgcaagttccactat
ctccccttcctgccctcaactgaggacgtttacgactgcagggtggagca
ctggggcttggatgagcctcttctcaagcactgggagtttgatgctccaa
gccctctcccagagactacagagaacgtggtgtgtgccctgggcctgact
gtgggtctggtgggcatcattattgggaccatcttcatcatcaagggatt
gcgcaaaagcaatgcagcagaacgcagggggcctctgtaa
Underlined: nucleotides encoding the signal peptide

One skilled in the art will appreciate that HLA-DRA nucleic acid and protein molecules can vary from those publicly available, such as polymorphisms resulting in one or more substitutions, deletions, insertions, or combinations thereof, while still retaining HLA-D A biological activity. Accordingly, in various embodiments, the amino acid sequence of the HLA-DRA component of the protein of the invention may be about 95%, about 96%, about 97%, about 98%, about 99% identical to the HLA-DRA sequence publicly available or to SEQ ID NO: 52, or fragment thereof. A fragment can be between 3-10 amino acids, 10-20 amino acids, 20-40 amino acids, 40-56 amino acids in length or even longer. Amino acid sequences having about 95%, about 96%, about 97%, about 98%, about 99% identity to the fragments described herein are also included within the scope of the present invention.

In various embodiments, the nucleic acid sequence encoding the HLA-DRA component of the protein of the invention may be about 95%, about 96%, about 97%, about 98%, about 99% identical to the HLA-DRA sequence publicly available or to SEQ ID NO: 53, or fragment thereof. A fragment can be between 3-10 nucleotides, 10-20 nucleotides, 20-40 nucleotides, 40-56 nucleotides in length or even longer. Nucleic acid sequences having about 95%, about 96%, about 97%, about 98%, about 99% identity to the fragments described herein are also included within the scope of the present invention.

In embodiments, the antigenic cancer peptide is 8-30 amino acids in length. For example, the antigenic cancer peptide is 8, 9, 10, 11, 12, 13, 14, 15, 16, 17, 18, 19, 20, 21, 22, 23, 24, 25, 26, 27, 28, 29, or 30 amino acids in length. For example, the antigenic cancer peptide is 8-15, 10-20, 15-24, or 20-30 amino acids in length. Typically, the antigenic cancer peptide is 15-24 amino acids in length.

In embodiments, the antigenic cancer peptide comprising, or consisting of, an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% of any one of SEQ ID Nos: 3, 5, 7, 9, 11, 13, 15, 17, 19, 21, 23, 25, 27, 29, 31, 33, 35, 37, 39, 41, 43, and 45.

In embodiments, the antigenic cancer peptide is encoded by a nucleic acid sequence comprising, or consisting of, a nucleic acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% of any one of SEQ ID Nos: 2, 4, 6, 8, 10, 12, 14, 16, 18, 20, 22, 24, 26, 28, 30, 32, 34, 36, 38, 40, 42, and 44.

In embodiments, one exemplary fusion protein (e.g., IDH1-R132Hshort construct) includes amino acid sequence:

(SEQ ID NO: 54)

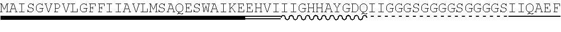

YLNPDQSGEFMFDFDGDEIFHVDMAKKETVWRLEEFGRFASFEAQGALANIAVDKANLEIMTKR
SNYTPITNDKFTPPVVNVTWLRNGKPVTTGVSETVFLPREDHLFRKFHYLPFLPSTEDVYDCRV
EHWGLDEPLLKHWEFDAPSPLPETTENVVCALGLTVGLVGIIIGTIFIIKGLRKSNAAERRGPL

Thick underline: HLA-DRA signal peptide
Double underline: 4 amino acids (protease recognition sequence of the signal peptide)
Wave underline: neoantigen IDH1-R132H
Dotted underline: linker
Underline: HLA-DRA

In embodiments, the fusion protein comprising, or consisting of, an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% of any one of SEQ ID No: 54.

Corresponding DNA sequence of the IDH1-R132Hshort construct includes:

(SEQ ID NO: 55)
5′-
atggccataagtggagtccctgtgctaggatttttcatcatagctgtgct
gatgagcgctcaggaatcatgggctatcaaagaagaacatgtgatcataa
tcggtcatcatgcttatggggaccagatcatcggaggaggaagtggcggc
ggcggcagcggtggtggtggttcgatcatccaggccgagttctatctgaa
tcctgaccaatcaggcgagtttatgtttgactttgatggtgatgagattt
tccatgtggatatggcaaagaaggagacggtctggcggcttgaagaattt
ggacgatttgccagctttgaggctcaaggtgcattggccaacatagctgt
ggacaaagccaacttggaaatcatgacaaagcgctccaactatactccga
tcaccaatgacaagttcaccccaccagtggtcaatgtcacgtggcttcga
aatggaaaacctgtcaccacaggagtgtcagagacagtcttcctgcccag
ggaagaccaccttttccgcaagttccactatctccccttcctgccctcaa
ctgaggacgtttacgactgcagggtggagcactggggcttggatgagcct
cttctcaagcactgggagtttgatgctccaagccctctcccagagactac
agagaacgtggtgtgtgccctgggcctgactgtgggtctggtgggcatca
ttattgggaccatcttcatcatcaagggattgcgcaaaagcaatgcagca
gaacgcagggggcctctgtaa

In embodiments, the fusion protein is encoded by a nucleic acid sequence comprising, or consisting of, a nucleic acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% of SEQ ID No: 55.

In embodiments, one exemplary fusion protein (e.g., IDH1-R132H construct) includes amino acid sequence:

(SEQ ID NO: 56)

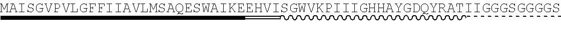

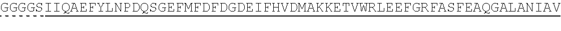

DKANLEIMTKRSNYTPITNDKFTPPVVNVTWLRNGKPVTTGVSETVFLPREDHLFRKFHYLPFL
PSTEDVYDCRVEHWGLDEPLLKHWEFDAPSPLPETTENVVCALGLTVGLVGIIIGTIFIIKGLR
KSNAAERRGP

Thick underline: HLA-DRA signal peptide
Double underline: 4 amino acids (protease recognition sequence of the signal peptide)
Wave underline: neoantigen IDH1-R132H
Dotted underline: linker
Underline: HLA-DRA

In embodiments, the fusion protein comprising, or consisting of, an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% of SEQ ID No: 56.

Corresponding DNA sequence of the IDH1-R132H construct includes:

(SEQ ID NO: 57)
5′-
atggccataagtggagtccctgtgctaggatttttcatcatagctgtgct
gatgagcgctcaggaatcatgggctatcaaagaagaacatgtgatcagcg
gatgggtaaaacctattataatcggtcatcatgcttatggggaccagtac
agagcaacgatcatcggaggaggaagtggcggcggcggcagcggtggtgg
tggttcgatcatccaggccgagttctatctgaatcctgaccaatcaggcg
agtttatgtttgactttgatggtgatgagattttccatgtggatatggca
aagaaggagacggtctggcggcttgaagaatttggacgatttgccagctt
tgaggctcaaggtgcattggccaacatagctgtggacaaagccaacttgg
aaatcatgacaaagcgctccaactatactccgatcaccaatgacaagttc
accccaccagtggtcaatgtcacgtggcttcgaaatggaaaacctgtcac
cacaggagtgtcagagacagtcttcctgcccagggaagaccaccttttcc
gcaagttccactatctccccttcctgccctcaactgaggacgtttacgac
tgcagggtggagcactggggcttggatgagcctcttctcaagcactggga
gtttgatgctccaagccctctcccagagactacagagaacgtggtgtgtg
ccctgggcctgactgtgggtctggtgggcatcattattgggaccatcttc
atcatcaagggattgcgcaaaagcaatgcagcagaacgcagggggcctct
gtaa

In embodiments, the fusion protein is encoded by a nucleic acid sequence comprising, or consisting of, a nucleic acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% of SEQ ID No: 57.

In embodiments, one exemplary fusion protein (e.g., IDH2-R140Q construct) includes amino acid sequence:

(SEQ ID NO: 58)

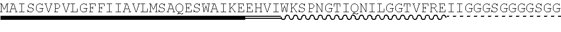

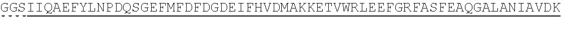

ANLEIMTKRSNYTPITNDKFTPPVVNVTWLRNGKPVTTGVSETVFLPREDHLFRKFHYLPFLPS
TEDVYDCRVEHWGLDEPLLKHWEFDAPSPLPETTENVVCALGLTVGLVGIIIGTIFIIKGLRKS
NAAERRGPL

Thick underline: HLA-DRA signal peptide
Double underline: 4 amino acids (protease recognition sequence of the signal peptide)
Wave underline: neoantigen IDH2-R140Q
Dotted underline: linker
Underline: HLA-DRA

In embodiments, the fusion protein comprising, or consisting of, an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% of SEQ ID No: 58.

Corresponding DNA sequence of the IDH2-R140Q construct includes:

(SEQ ID NO: 59)
5′-
atggccataagtggagtccctgtgctaggatttttcatcatagctgtgct
gatgagcgctcaggaatcatgggctatcaaagaagaacatgtgatctgga
agtctccgaacggcaccattcagaatattctcggcggcactgtgttccgg
gagatcatcggaggaggaagtggcggcggcggcagcggtggtggtggttc
gatcatccaggccgagttctatctgaatcctgaccaatcaggcgagttta
tgtttgactttgatggtgatgagattttccatgtggatatggcaaagaag
gagacggtctggcggcttgaagaatttggacgatttgccagctttgaggc
tcaaggtgcattggccaacatagctgtggacaaagccaacttggaaatca
tgacaaagcgctccaactatactccgatcaccaatgacaagttcacccca
ccagtggtcaatgtcacgtggcttcgaaatggaaaacctgtcaccacagg
agtgtcagagacagtcttcctgcccagggaagaccaccttttccgcaagt
tccactatctccccttcctgccctcaactgaggacgtttacgactgcagg
gtggagcactggggcttggatgagcctcttctcaagcactgggagtttga
tgctccaagccctctcccagagactacagagaacgtggtgtgtgccctgg
gcctgactgtgggtctggtgggcatcattattgggaccatcttcatcatc
aagggattgcgcaaaagcaatgcagcagaacgcagggggcctctgtaa

In embodiments, the fusion protein is encoded by a nucleic acid sequence comprising, or consisting of, a nucleic acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% of SEQ ID No: 59.

In embodiments, one exemplary fusion protein (e.g., IDH2-R172K construct) includes amino acid sequence:

(SEQ ID NO: 60)

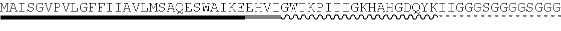

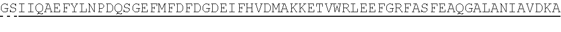

NLEIMTKRSNYTPITNDKFTPPVVNVTWLRNGKPVTTGVSETVFLPREDHLFRKFHYLPFLPST
EDVYDCRVEHWGLDEPLLKHWEFDAPSPLPETTENVVCALGLTVGLVGIIIGTIFIIKGLRKSN
AAERRGPL

Thick underline: HLA-DRA signal peptide
Double underline: 4 amino acids (protease recognition sequence of the signal peptide)
Wave underline: neoantigen IDH2-R172K
Dotted underline: linker
Underline: HLA-DRA

In embodiments, the fusion protein comprising, or consisting of, an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% of SEQ ID No: 60.

Corresponding DNA sequence of the IDH2-R172K construct includes:

(SEQ ID NO: 61)
5′-
atggccataagtggagtccctgtgctaggatttttcatcatagctgtgct
gatgagcgctcaggaatcatgggctatcaaagaagaacatgtgatcggtt
ggacgaaaccaatcactattggcaagcatgctcatggagaccagtacaag
atcatcggaggaggaagtggcggcggcggcagcggtggtggtggttcgat
catccaggccgagttctatctgaatcctgaccaatcaggcgagtttatgt
ttgactttgatggtgatgagattttccatgtggatatggcaaagaaggag
acggtctggcggcttgaagaatttggacgatttgccagctttgaggctca
aggtgcattggccaacatagctgtggacaaagccaacttggaaatcatga
caaagcgctccaactatactccgatcaccaatgacaagttcaccccacca
gtggtcaatgtcacgtggcttcgaaatggaaaacctgtcaccacaggagt
gtcagagacagtcttcctgcccagggaagaccaccttttccgcaagttcc
actatctccccttcctgccctcaactgaggacgtttacgactgcagggtg
gagcactggggcttggatgagcctcttctcaagcactgggagtttgatgc
tccaagccctctcccagagactacagagaacgtggtgtgtgccctgggcc
tgactgtgggtctggtgggcatcattattgggaccatcttcatcatcaag
ggattgcgcaaaagcaatgcagcagaacgcagggggcctctgtaa

In embodiments, the fusion protein is encoded by a nucleic acid sequence comprising, or consisting of, a nucleic acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% of SEQ ID No: 61.

In embodiments, the fusion protein of the invention includes:
a. a signal peptide comprising, or consisting of, an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% of SEQ ID NO: 62;
b. an antigenic cancer peptide comprising, or consisting of, an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% of any one of SEQ ID Nos: 3, 5, 7, 9, 11, 13, 15, 17, 19, 21, 23, 25, 27, 29, 31, 33, 35, 37, 39, 41, 43, and 45; and
c. a linker comprising, or consisting of, an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% of any one of SEQ ID Nos: 46-50.

In embodiments, the fusion protein of the invention is partially encoded by a nucleic acid sequence comprising:
a. a nucleic acid sequence encoding signal peptide that comprises, or consists of, an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% of SEQ ID NO: 62;
b. a nucleic acid sequence encoding an antigenic cancer peptide that comprises, or consists of, an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% of any one of SEQ ID Nos: 3, 5, 7, 9, 11, 13, 15, 17, 19, 21, 23, 25, 27, 29, 31, 33, 35, 37, 39, 41, 43, and 45; and
c. a nucleic acid sequence encoding a linker that comprises, or consists of, an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% of any one of SEQ ID Nos: 46-50.

Another aspect provided herein pertains to an antigen-presenting cell which is expressing one or more proteins described herein. Typically, the protein is expressed at the cell surface of the antigen-presenting cell. In embodiments, the antigen-presenting cell is a dendritic cell or B cell. Typically, the antigen-presenting cell is a dendritic cell. The antigen-presenting cell can be derived from a single subject or from multiple subjects. Typically, the antigen-presenting cell is obtained from the same subject who will receive the treatment. Thus, the antigen-presenting cell can be autologous to the recipient.

The one or more proteins can be expressed in an antigen-presenting cell according to any method available in the art, such as, via conventional transformation, transduction, infection or transfection techniques. As used herein, the terms “transformation,” “transduction”, “infection” and “transfection” are intended to refer to a variety of art recognized techniques for introducing foreign nucleic acid (e.g., DNA) into a host cell, including calcium phosphate or calcium chloride co precipitation, DEAE dextran mediated transfection, lipofection, or electroporation. In addition transfection can be mediated by a transfection agent. By “transfection agent” is meant to include any compound that mediates incorporation of DNA in the host cell, e.g., liposome. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

In yet another aspect, a dendritic cell or B cell that includes any nucleic acid as described herein is provided.

Also provided herein are nucleic acids (polynucleotide sequences) encoding one or more proteins described herein. A “polynucleotide” is a nucleic acid polymer of ribonucleic acid (RNA), deoxyribonucleic acid (DNA), modified RNA or DNA, or RNA or DNA mimetics (such as PNAs), and derivatives thereof, and homologues thereof. Thus, polynucleotides include polymers composed of naturally occurring nucleobases, sugars and covalent inter-nucleoside (backbone) linkages as well as polymers having non-naturally-occurring portions that function similarly. Such modified or substituted nucleic acid polymers are well known in the art and for the purposes of the present invention, are referred to as “analogues.” Typically, a nucleic acid is an mRNA. Alternatively, a nucleic acid is a dsDNA.

In embodiments, the nucleic acid described herein forms part of a vector nucleic acid. Typically, the vector is a replication-incompetent viral vector. For example, the replication-incompetent viral vector is a replication-incompetent DNA viral vector (including, but is not limited to, adenoviruses, adeno-associated viruses). For example, the replication-incompetent viral vector is a replication-incompetent RNA viral vector (including, but is not limited to, replication defective retroviruses and lentiviruses).

In embodiments, the polypeptides and other compositions of the invention are isolated or purified. As used herein, an “isolated” or “purified” nucleotide or polypeptide is substantially free of other nucleotides and polypeptides. Purified nucleotides and polypeptides are also free of cellular material or other chemicals when chemically synthesized. Purified compounds are at least about 60% by weight (dry weight) the compound of interest. Preferably, the preparation is at least about 75%, more preferably at least about 90%, and most preferably at least about 99%, by weight the compound of interest. For example, a purified nucleotides and polypeptides is one that is at least about 90%, about 91%, about 92%, about 93%, about 94%, about 95%, about 98%, about 99%, or about 100% (w/w) of the desired oligosaccharide by weight. Purity is measured by any appropriate standard method, for example, by column chromatography, thin layer chromatography, or high-performance liquid chromatography (HPLC) analysis. The nucleotides and polypeptides are purified and used in a number of products for consumption by humans as well as animals, such as companion animals (dogs, cats) as well as livestock (bovine, equine, ovine, caprine, or porcine animals, as well as poultry). “Purified” also defines a degree of sterility that is safe for administration to a human subject, e.g., lacking infectious or toxic agents.

Similarly, by “substantially pure” is meant a nucleotide or polypeptide that has been separated from the components that naturally accompany it. Typically, the nucleotides and polypeptides are substantially pure when they are at least about 60%, about 70%, about 80%, about 90%, about 95%, or even about 99%, by weight, free from the proteins and naturally-occurring organic molecules with they are naturally associated.

In embodiments, any nucleic acid described herein can be within a pharmaceutically acceptable liposomal construct or a pharmaceutically acceptable polymeric construct.

Liposomes, also known as vesicles, are generally composed of phospholipids and other lipid components such as cholesterol. They can function as carriers whose essential structural feature is a bipolar lipid membrane which envelops an aqueous core volume in which pharmacological agents are solubilized and therefore encapsulated. Various lipid formulations and methods for their preparation have been described for the delivery of pharmaceutically active agents to a host. For example, Geho and Lau in U.S. Pat. No. 4,603,044 describe a targeted liposomal delivery system for delivery of a drug to the hepatobiliary receptors of the liver. The system is composed of a drug or diagnostic agent encapsulated in or associated with lipid membrane structures in the form of vesicles or liposomes, and a molecule having a fatty substituent attached to the vesicle wall and a target substituent which is a biliary attracted chemical, such as a substituted iminodiacetate complex. The system is particularly useful for the delivery of insulin and serotonin in the treatment of Types I and II diabetes, respectively.

Various polymeric formulations of biologically active agents and methods for their preparation have been described. U.S. Pat. Nos. 3,773,919, 3,991,776, 4,076,779, 4,093,709, 4,118,470, 4,131,648, 4,138,344, 4,293,539 and 4,675,189, inter alia, disclose the preparation and use of biocompatible, biodegradable polymers, such as poly (lactic acid), poly(glycolic acid), copolymers of glycolic and lactic acids, poly (o-hydroxycarboxy lie acid), polylactones, polyacetals, polyorthoesters and polyorthocarbonates, for the encapsulation of drugs and medicaments. These polymers mechanically entrap the active constituents and later provide controlled release of the active ingredient via polymer dissolution or degradation. Certain condensation polymers formed from divinyl ethers and polyols are described in Polymer Letters, 18, 293 (1980). Polymers have proven to be successful controlled-release drug delivery devices.

More information about liposomal constructs or polymeric constructs that can be used for the present invention can be found at Schwendener R A et al., Ther Adv Vaccines. 2014 November; 2(6): 159-182; Li Yet al., J Gene 2011, Med 13: 60-72; Pichon C et al., Methods Mol Biol 2013 969: 247-274; McNamara M A et al., J Immunol Res. 2015; 2015: 794528; Sayour E. J. et al., Journal for Immunotherapy of Cancer. 2015;3, article 13; Bettinger T. et al, Current Opinion in Molecular Therapeutics. 2001;3(2):116-124; Lu D. et al., Cancer Gene Therapy. 1994;1(4):245-252; Wasungu L. et al., Journal of Controlled Release. 2006;116(2):255-264; Little S. et al., Proceedings of the National Academy of Sciences of the United States of America. 2004;101(26):9534-9539; Phua K. et al., Journal of Controlled Release. 2013;166(3):227-233; Su X et al., Molecular Pharmaceutics. 2011;8(3):774-787; Phua K. K. L. et al., Nanoscale. 2014;6(14):7715-7729; Phua K. K. L. et al., Scientific Reports. 2014; 4, article 5128.

The present invention also provides pharmaceutical compositions/formulations that include a nucleic acid disclosed herein in combination with at least one pharmaceutically acceptable excipient or carrier.

Acceptable carriers, excipients or stabilizers are nontoxic to recipients at the dosages and concentrations employed, and include buffers such as phosphate, citrate, or acetate at a pH typically of 5.0 to 8.0, most often 6.0 to 7.0; salts such as sodium chloride, potassium chloride, etc. to make isotonic; antioxidants, preservatives, low molecular weight polypeptides, proteins, hydrophilic polymers such as polysorbate 80, amino acids such as glycine, carbohydrates, chelating agents, sugars, and other standard ingredients known to those skilled in the art (Remington's Pharmaceutical Science 16^{th} edition, Osol, A. Ed. 1980).

A pharmaceutical formulation including a nucleic acid as described herein can be administered by a variety of methods known in the art. The route and/or mode of administration vary depending upon the desired results. In embodiments, administration is intravenous, intramuscular, intraperitoneal, or subcutaneous, or administered proximal to the site of the target. Pharmaceutically acceptable excipients can be suitable for intravenous, intramuscular, subcutaneous, parenteral, spinal or epidermal administration (e.g., by injection or infusion).

Pharmaceutical formulations of the nucleic acid as described herein can be prepared in accordance with methods well known and routinely practiced in the art. See, e.g., Remington: The Science and Practice of Pharmacy, Mack Publishing Co., 20^{th} ed., 2000; and Sustained and Controlled Release Drug Delivery Systems, J. R. Robinson, ed., Marcel Dekker, Inc., New York, 1978. Pharmaceutical compositions are preferably manufactured under GMP conditions.

Actual dosage levels of the active ingredients (i.e., the nucleic acids described herein) in the pharmaceutical compositions of the present invention can be varied so as to obtain an amount of the active ingredient which is effective to achieve the desired therapeutic response for a particular patient, composition, and mode of administration, without being toxic to the patient. The selected dosage level depends upon a variety of pharmacokinetic factors including the activity of the particular compositions of the present invention employed, the route of administration, the time of administration, the rate of excretion of the particular composition (e.g., the nucleic acid described herein) being employed, the duration of the treatment, other drugs, compounds and/or materials used in combination with the particular compositions employed, the age, sex, weight, condition, general health and prior medical history of the patient being treated, and like factors.

A physician or veterinarian can start doses of the nucleic acid of the invention employed in the pharmaceutical formulation at levels lower than that required to achieve the desired therapeutic effect and gradually increase the dosage until the desired effect is achieved. In general, effective doses of the compositions of the present invention vary depending upon many different factors, including the specific disease or condition to be treated, means of administration, target site, physiological state of the patient, whether the patient is human or an animal, other medications administered, and whether treatment is prophylactic or therapeutic. Treatment dosages need to be titrated to optimize safety and efficacy. For administration with a pharmaceutical formulation of the invention, the dosage ranges from about 0.0001 to 100 mg/kg, and more usually 0.01 to 5 mg/kg, of the host body weight. For example dosages can be 1 mg/kg body weight or 10 mg/kg body weight or within the range of 1-10 mg/kg. An exemplary treatment regime entails administration once per every two weeks or once a month or once every 3 to 6 months.

The compositions provided herein can be administered on multiple occasions. Intervals between single dosages can be weekly, monthly or yearly. Intervals can also be irregular as indicated by measuring immune response to the neoantigen. Alternatively, composition can be administered as a sustained release formulation, in which case less frequent administration is required. Dosage and frequency vary depending on the half-life of the composition in the patient. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

### III. Methods

In one aspect, a method of treating cancer in a patient in need thereof is provided. The method includes contacting in vitro the antigen-presenting cell as described herein with a CD4+ T cell, thereby activing the CD4+ T cell, where the CD4+ T cell and the antigen-presenting cell are derived from the patient; allowing the activated CD4+ T cell to expand thereby forming a plurality of expanded CD4+ T cells; and administering to the patient an effective amount of the plurality of expanded CD4+ T cells. In embodiments, the cancer is metastatic glioblastoma multiforme (GBM).

In one aspect, a method of treating cancer in a patient in need thereof is provided. The method includes administering to the patient an effective amount of the nucleic acid as described herein. In embodiments, the nucleic acid is an mRNA. In embodiments, the nucleic acid forms part of a nucleic acid-liposome complex. In embodiments, the effective amount is effective to activate CD4+ T cells within the patient. In embodiments, the nucleic acid forms part of a replication-incompetent viral vector.

Isolation and activation of CD4+ T cells can be determined according to any method known in the art. For example, whole blood (about 20 ml) is drawn from a subject in an EDTA-containing blood tube. Under sterile conditions the blood sample is diluted to 1:1 with PBS, layered on top of 30 ml of Ficoll-hypaque and spin at 400 g for 30 min. The peripheral blood lymphocytes layer is collected and diluted with PBS (1:7) followed by the centrifugation at 300 g for 10 min. Cells are rinsed twice more by resuspension in PBS. Viable cells are counted by Trypan blue exclusion. Adherent cells are removed to enrich for T cells with sterile nylon wool columns. Briefly, the nylon wool column was washed with T-cell medium (1640 RPMI with 10% FBS) and incubated at 37° C. for 1 h. Peripheral blood lymphocytes (10⁷ cells ml⁻¹) were added to the pre-warmed column and incubated for 1 h at 37° C., after which non-adherent cells were collected by opening the column's stopcock. The column was washed twice with 5 ml of T-cell medium to collect total effluent. T-cell-enriched fractions were incubated with carboxyfluorescein diacetate succinimidyl ester (CFSE) solution (4 μM in PBS) for 8 min at 37° C. water bath. After incubation, the solution was diluted with 10 volumes of T-cell medium, pelleted by centrifugation (300 g, 10 min) and washed twice with PBS containing 0.1% bovine serum albumin. CFSE-labeled cells were resuspended, counted and a fraction was used to evaluate CFSE fluorescence by flow cytometry (Accuri division of BD Biosciences, San Jose, Calif., USA). Prior to flow cytometry cells were immunostained with Alexa647-conjugated anti-CD4 antibody (BD Biosciences), to allow for analysis of CFSE fluorescence in the CD4⁺ population. Further flow cytometric data and histogram analyses were done using FCS express 4 flow cytometry software (De Novo Software, Los Angeles, Calif., USA).

### Embodiments

Embodiments contemplated herein include embodiments P1 to PX28 following.

### Embodiment P1

A protein comprising an antigenic cancer peptide covalently attached to a mature MHC class II peptide, said antigenic cancer peptide capable of non-covalently binding directly to said MHC class II peptide.

### Embodiment P2

The protein of embodiment P1, wherein said antigenic cancer peptide is N-terminal to said mature MHC class II peptide.

### Embodiment P3

The protein of one of embodiments P1 or P2, further comprising a peptide linker covalently linking said antigenic cancer peptide and said mature MHC class II peptide.

### Embodiment P4

The protein of embodiment P3, wherein said peptide linker consists of one or more glycine amino acids, one or more serine amino acids, or a combination of one or more glycine and one or more serine amino acids.

### Embodiment P5

The protein of one of embodiments P1 to P4, further comprising a signal peptide covalently attached to the N-terminus of said antigenic cancer peptide.

### Embodiment P6

The protein of one of embodiments P1 to P5, wherein said mature MHC class II peptide is a mature HLA-DRA alpha chain peptide.

### Embodiment P7

The protein of one of embodiments P1 to P6, wherein said antigenic cancer peptide is 8-30 amino acids in length.

### Embodiment P8

The protein of one of embodiments P1 to P6, wherein said antigenic cancer peptide is 15-24 amino acids in length.

### Embodiment P9

An antigen-presenting cell comprising the protein of one of embodiments P1 to P8.

### Embodiment P10

The antigen-presenting cell of embodiment P9, wherein said protein is at the cell surface of said antigen-presenting cell.

### Embodiment P11

The antigen-presenting cell of embodiment P9 or P10, wherein said antigen-presenting cell is a dendritic cell or B cell.

### Embodiment P12

The antigen-presenting cell of embodiment P9 or P10, wherein said antigen-presenting cell is a dendritic cell.

### Embodiment P13

A nucleic acid encoding the protein of one of embodiments P1 to P8.

### Embodiment P14

The nucleic acid of embodiment P13, wherein said nucleic acid is an mRNA.

Embodiment P15

The nucleic acid of embodiment P13, wherein said nucleic acid is a dsDNA.

### Embodiment P16

The nucleic acid of embodiment P13, wherein said nucleic acid forms part of a replication-incompetent viral vector nucleic acid.

### Embodiment P17

The nucleic acid of embodiment P16, wherein said a replication-incompetent viral vector nucleic acid is a replication-incompetent lentiviral vector.

### Embodiment P18

The nucleic acid of embodiment P16, wherein said replication-incompetent viral vector nucleic acid is a replication-incompetent DNA viral vector or replication-incompetent RNA viral vector.

### Embodiment P19

The nucleic acid of one of embodiments P13 to P18, wherein said nucleic acid is within a pharmaceutically acceptable liposomal construct or pharmaceutically acceptable polymeric construct.

### Embodiment P20

A pharmaceutical formulation comprising the nucleic acid of one of embodiments P13-P19 and a pharmaceutically acceptable excipient.

### Embodiment P21

A dendritic cell or B cell comprising the nucleic acid of one of embodiments P13-P19.

### Embodiment P22

A method of treating cancer in a patient in need thereof, the method comprising: (i) contacting in vitro the antigen-presenting cell of one of embodiments P9-P12 with a CD4+ T cell, thereby activating said CD4+ T cell, wherein the CD4+ T cell and the antigen-presenting cell are derived from said patient; (ii) allowing said CD4+ T cell to expand thereby forming a plurality of expanded CD4+ T cells; and (iii) administering to said patient an effective amount of said plurality of expanded CD4+ T cells.

### Embodiment P23

The method of embodiment P22, wherein said cancer is metastatic glioblastoma multiforme (GBM).

### Embodiment P24

A method of treating cancer in a patient in need thereof, the method comprising administering to said patient an effective amount of the nucleic acid of one of embodiments P13-P19.

### Embodiment P25

The method of embodiment P24, wherein said nucleic acid is an mRNA.

### Embodiment P26

The method of embodiment P24 or P25, wherein said nucleic acid forms part of a nucleic acid-liposome complex.

### Embodiment P27

The method of one of embodiments P24-P26, wherein said effective amount is effective to activate CD4+ T cells within said patient.

### Embodiment P28

The method of embodiment P24, wherein said nucleic acid forms part of a replication-incompetent viral vector.

### EXAMPLES

### Example 1: CD4see Construct Design and Creation

HLA-DRA1 sequence (SEQ ID NO: 52 or 53) was downloaded from NCBI nucleotide database. Oligonucleotides encoding primers were designed for the amplification of the coding sequence (cDNA) for HLA-DRA using polymerase chain reaction (PCR). HLA-DRA1 cDNA was produced by PCR using reverse transcribed RNA template from the inventor's blood. The cDNA was cloned into a plasmid vector.

A glycine/serine linker sequence was designed and incorporated immediately after the signal peptide cleavage site of HLA-DRA1 cDNA using site-directed mutagenesis. All constructs are derivatives of this base vector and each construct incorporates a different target peptide immediately after the linker sequence. The DNA and RNA sequences of target peptides for CD4see are derived from proteins identified as either driver mutations or passenger mutations.

CD4see constructs were designed and produced that target neoantigen driver mutations in metastatic glioblastoma multiforme (GBM). More than 70% of recurrent GBM cells carry mutations in isocitrate dehydrogenase (IDH)-1 or IDH2. Mutations at amino acid arginine 172 of IDH2 can alter the enzymatic activity of the protein. The most common IDH1 mutations found in later stage GBM are a single amino acid missense mutation in IDH1 at arginine 132 (R132) which change the amino acid sequence of the IDH1 protein. CD4see constructs that include the region of these mutations were designed and generated.

Complementary oligonucleotides that encode IDH1 neoantigen peptide were ordered from a commercial supplier, heated, annealed and ligated into the prepared CD4see construct. Specifically, CD4see construct was designed and constructed to have a Bcl1 restriction endonuclease site between the linker and the non-signal peptide region of HLA-DRA1. The annealed IDH1 oligonucleotides include ends that are compatible for ligation into Bcl1 cut sites. In preparation for the ligation, the CD4see construct was prepared by digesting a plasmid containing CD4see with Bcl1 enzyme. The CD4see construct generated by the ligation reactions introduces coding sequence for the IDH1 neoantigen peptide within the CD4see backbone between the signal peptide and the linkers peptide using site-directed mutagenesis, in frame.

### Example 2: In Vitro Human Testing

The ability of CD4see constructs to stimulate the proliferation of antigen-specific CD4+ T cells in normal healthy adults was tested using white blood cells drawn from a healthy volunteer. Expression plasmids containing CD4see constructs were grown in bacteria and purified using commercial plasmid purification kits that remove endotoxin contaminants. Whole blood drawn from the volunteer was fractionated to purify monocytes. The monocytes were transiently transfected with plasmid DNA containing a CD4see construct, and the cells were stimulated with lipopolysaccharide and tumor necrosis factor-α (TNF-α) for 1-3 days. T cells isolated from the same blood sample (histocompatible match) were labeled with a fluorescent probe such as CFSE (Begum et al., 2014). After 3 days of stimulation treated monocytes were then co-cultured with fluorescently labeled T cells for an additional 7 days. The cell mixture was immunostained with fluorescently tagged anti-CD4 antibodies, and assayed by flow cytometry. Flow cytometer gates were set to identify viable cells using a forward scatter (FSC) and side scatter (SSC) plot and those cells were then gated for the anti-CD4 fluorophore to select the CD4+cells. CFSE fluorescence intensity of the CD4+population was plotted on a histogram to reveal the proportion of CD4+ T cells that underwent 5-8 or more cell divisions during the co-culture period. Cell divisions were calculated by CFSE fluorescence in a companion co-culture that contained IL-2 without a CD4see construct. CD4see constructs that stimulate strong CD4+ T cells proliferation in several human subjects are developed further.

### Example 3: Use of CD4see to Treat Cancer

A patient with metastatic colorectal cancer is treated with CD4see technology as follows. First the patient has a biopsy take of 1-3 tumors and RNA is isolated using one of several commercially available RNA purification kits, such as Qiagen's RNeasy. RNA is processed for next generation RNA sequencing (RNAseq) using any of several technologies available from Illumina, Roche or other providers. RNAseq runs that generate 10-30 million reads will provide adequate coverage of mRNA expressed by the tumor(s). The sequence is analyzed to identify single nucleotide polymorphisms that produce in-frame amino acid mutations resulting in neoantigens (Polyakova et al., 2015). These neoantigens include driver mutations that facilitate tumor growth or passenger mutations that do not appear to facilitate tumor growth. Examples of known driver mutations may include but are not limited to IDH1-R132H, IDH2-R140Q, IDH2-R172K, BRAF-V600E, KRAS-G12D, NRAS-Q61K, PIK3CA-E545K. RNAseq also detects higher expression of cancer-testis (CT) antigens that frequently accompany tumor growth, such as NY-ESO-1 and MAGE-A3.

Analysis of neoantigens, both driver and passenger mutations, and elevated CT antigens is done to identify the most suitable targets for CD4see mediated responses. Target peptides are produced, which includes selected mutations and 8-10 flanking residues amino and carboxyl to that site in the affected protein sequence. Oligonucleotides that encode those sequences are produced, hybridized, and spliced into a CD4see vector such that the neoantigen sequence is translated in-frame with the CD4see protein. If necessary, the completed CD4see constructs are transferred into other expression vectors. This same procedure is used to generate 8-20 or more neoantigen CD4see constructs for the patient. In instances where a common driver mutation is required, previously developed constructs may be used.

Delivery of CD4see constructs to stimulate neoantigen-specific CD4+ T cells

In one iteration mRNA encoding CD4see-neoantigenic peptide constructs will be transcribed in vitro. CD4see-neoantigen constructs may be transcribed individually or batched together. The mRNA produced is purified from DNA and other impurities. The purified mRNA is packaged into liposomes and used to transfect antigen-presenting cells (APC) derived from the patient or a suitable donor. Alternatively, mRNA or expression plasmids containing CD4see-neoantigen constructs are introduced into APC's by electroporation. For example, 2 million APC's prepared from the patient's own blood monocytes are placed in a cuvette with a suitable amount of mRNA or expression plasmid and subjected to an electronic pulse of sufficient strength and duration for mRNA to transfected into the cells. A second expression plasmid containing a fluorescent reported protein such as DS-RED or M-CHERRY may be used to establish transfection efficiency. Treated APC's are co-cultured with T cells isolated from the patient's blood for such as an appropriate number of neoantigen specific CD4+ T cells have been produced. Neoantigen-specific CD4+ T cells will be purified and returned to the patient by an intravenous drip.

Another iteration may involve administering mRNA transduced APC's to the patient by intravenous drip or injection into lymph nodes. In vivo administration of APC's is supplemented with a peptide vaccine that incorporates the targeted neoantigen sequences so as to facilitate B-cell/antibody responses stimulated by CD4see responsive Th2 cells.

Another iteration may involve integrating CD4see-neoantigen constructs into lentiviral vector that will be used to express the protein in APC's for in vitro or in vivo therapy as described above.

Another iteration may use in vitro studies to screen all possible constructs for the most responsive combinations for use in the patient.

Another iteration may use tumor infiltrating lymphocytes (TILs) as the source of T cells in APC-mediated proliferation.

What is claimed is:

1. A protein comprising an antigenic cancer peptide covalently attached to a mature MHC class II peptide, said antigenic cancer peptide capable of non-covalently binding directly to said MHC class II peptide.

2. The protein of claim 1, wherein said antigenic cancer peptide is N-terminal to said mature MHC class II peptide.

3. The protein of claim 1, further comprising a peptide linker covalently linking said antigenic cancer peptide and said mature MHC class II peptide.

4. The protein of claim 3, wherein said peptide linker consists of one or more glycine amino acids, one or more serine amino acids, or a combination of one or more glycine and one or more serine amino acids.

5. The protein of claim 1, further comprising a signal peptide covalently attached to the N-terminus of said antigenic cancer peptide.

6. The protein of claim 1, wherein said mature MHC class II peptide is a mature HLA-DRA alpha chain peptide.

7. The protein of claim 1, wherein said antigenic cancer peptide is 8-30 amino acids in length.

8. The protein of claim 1, wherein said antigenic cancer peptide is 15-24 amino acids in length.

9. An antigen-presenting cell comprising the protein of claim 1.

10. The antigen-presenting cell of claim 9, wherein said protein is at the cell surface of said antigen-presenting cell.

11. The antigen-presenting cell of claim 9, wherein said antigen-presenting cell is a dendritic cell or B cell.

12. The antigen-presenting cell of claim 9, wherein said antigen-presenting cell is a dendritic cell.

13. A nucleic acid encoding the protein of one of claims 1 to 8.

14. The nucleic acid of claim 13, wherein said nucleic acid is an mRNA.

15. The nucleic acid of claim 13, wherein said nucleic acid is a dsDNA.

16. The nucleic acid of claim 13, wherein said nucleic acid forms part of a replication-incompetent viral vector nucleic acid.

17. The nucleic acid of claim 16, wherein said a replication-incompetent viral vector nucleic acid is a replication-incompetent lentiviral vector.

18. The nucleic acid of claim 16, wherein said replication-incompetent viral vector nucleic acid is a replication-incompetent DNA viral vector or replication-incompetent RNA viral vector.

19. The nucleic acid of claim 13, wherein said nucleic acid is within a pharmaceutically acceptable liposomal construct or pharmaceutically acceptable polymeric construct.

20. A pharmaceutical formulation comprising the nucleic acid of claim 13 and a pharmaceutically acceptable excipient.

21. A dendritic cell or B cell comprising the nucleic acid of claim 13.

22. A method of treating cancer in a patient in need thereof, the method comprising:
(i) contacting in vitro the antigen-presenting cell of one of claims 9-12 with a CD4+ T cell, thereby activating said CD4+ T cell, wherein the CD4+ T cell and the antigen-presenting cell are derived from said patient;
(ii) allowing said CD4+ T cell to expand thereby forming a plurality of expanded CD4+ T cells; and
(iii) administering to said patient an effective amount of said plurality of expanded CD4+ T cells.

23. The method of claim 22, wherein said cancer is metastatic glioblastoma multiforme (GBM).

24. A method of treating cancer in a patient in need thereof, the method comprising administering to said patient an effective amount of the nucleic acid of claim 13.

25. The method of claim 24, wherein said nucleic acid is an mRNA.

26. The method of claim 24, wherein said nucleic acid forms part of a nucleic acid-liposome complex.

27. The method of claim 24, wherein said effective amount is effective to activate CD4+ T cells within said patient.

28. The method of claim 24, wherein said nucleic acid forms part of a replication-incompetent viral vector.
